SEQUENCE LISTING (54 sequences)

SEQ ID NO: 1 - DNA, 43 bp, Artificial Sequence
  synthetic probe
cgcgatctcgtcattggcggatcaaacggcctgcacgatcgcg

SEQ ID NO: 2 - DNA, 44 bp, Artificial Sequence
  synthetic probe
cgcgatcttggttgatacacctgaaacaaagcatcctgatcgcg

SEQ ID NO: 3 - DNA, 37 bp, Artificial Sequence
  synthetic probe
cgcgatcctgattcaggttacggacaaggtgatcgcg

SEQ ID NO: 4 - DNA, 43 bp, Artificial Sequence
  synthetic probe
cgcgatcccaggaagacaggtacaggatcattctgcgatcgcg

SEQ ID NO: 5 - DNA, 23 bp, Artificial Sequence
  synthetic primer
cgcatgacccaagggcaaagcgn

SEQ ID NO: 6 - DNA, 35 bp, Artificial Sequence
  synthetic nucleotide
gtcaaaaatcatgaacctcattacttatgataagn

SEQ ID NO: 7 - DNA, 35 bp, Artificial Sequence
  synthetic nucleotide
atttcatatatgtaattcctccacatctcattaan

SEQ ID NO: 8 - DNA, 33 bp, Artificial Sequence
  synthetic nucleotide
cactttttattcttcaaagatttgagctaattn

SEQ ID NO: 9 - DNA, 35 bp, Artificial Sequence
  synthetic nucleotide
ctctgctttatattataaaattacggctgaaatan

SEQ ID NO: 10 - DNA, 33 bp, Artificial Sequence
  synthetic nucleotide
tggaaatccatctctactttattgttttcttcn

SEQ ID NO: 11 - DNA, 34 bp, Artificial Sequence
  synthetic nucleotide
tccatctctactttattgttttcttcaaatattn

SEQ ID NO: 12 - DNA, 26 bp, Artificial Sequence
  synthetic oligonucleotide
aagcgattgatggtgatacggttaan

SEQ ID NO: 13 - DNA, 26 bp, Artificial Sequence
  synthetic oligonucleotide
aaatgcacttgcttcaggaccatatn

SEQ ID NO: 14 - DNA, 29 bp, Artificial Sequence
  synthetic oligonucleotide
ggtgaagatataccaagtgattatccatn

SEQ ID NO: 15 - DNA, 26 bp, Artificial Sequence
  synthetic oligonucleotide
gtgaggtgcgttaatattgccattan

SEQ ID NO: 16 - DNA, 28 bp, Artificial Sequence
  synthetic oligonucleotide
gcctcagtatgctcccacgaccgaattn

SEQ ID NO: 17 - DNA, 28 bp, Artificial Sequence
  synthetic oligonucleotide
tcagcagtttgcacatcaggcgaataan

SEQ ID NO: 18 - DNA, 17 bp, Artificial Sequence
  synthetic oligonucleotide
cgcatgacccaagggca

SEQ ID NO: 19 - DNA, 29 bp, Artificial Sequence
  synthetic oligonucleotide
atttcatatatgtaattcctccacatctc

SEQ ID NO: 20 - DNA, 29 bp, Artificial Sequence
  synthetic oligonucleotide
gtcaaaaatcatgaacctcattacttatg

SEQ ID NO: 21 - DNA, 29 bp, Artificial Sequence
  synthetic oligonucleotide
ctctgctttatattataaaattacggctg

SEQ ID NO: 22 - DNA, 27 bp, Artificial Sequence
  synthetic oligonucleotide
cactttttattcttcaaagatttgagc

SEQ ID NO: 23 - DNA, 27 bp, Artificial Sequence
  synthetic oligonucleotide
tggaaatccatctctactttattgttt

SEQ ID NO: 24 - DNA, 28 bp, Artificial Sequence
  synthetic oligonucleotide
tccatctctactttattgttttcttcaa

SEQ ID NO: 25 - DNA, 20 bp, Artificial Sequence
  synthetic oligonucleotide
aagcgattgatggtgatacg

SEQ ID NO: 26 - DNA, 20 bp, Artificial Sequence
  synthetic oligonucleotide
aaatgcacttgcttcaggac

SEQ ID NO: 27 - DNA, 23 bp, Artificial Sequence
  synthetic oligonucleotide
ggtgaagatataccaagtgatta

SEQ ID NO: 28 - DNA, 20 bp, Artificial Sequence
  synthetic oligonucleotide
gtgaggtgcgttaatattgc

SEQ ID NO: 29 - DNA, 29 bp, Artificial Sequence
  synthetic oligonucleotide
gaaccaacgcatgacccaagggcaaagcg

SEQ ID NO: 30 - DNA, 40 bp, Artificial Sequence
  synthetic oligonucleotide
tccaatatttcatatatgtaattcctccacatctcattaa

SEQ ID NO: 31 - DNA, 40 bp, Artificial Sequence
  synthetic oligonucleotide
aactatgtcaaaaatcatgaacctcattacttatgataag

SEQ ID NO: 32 - DNA, 40 bp, Artificial Sequence
  synthetic oligonucleotide
aataaactctgctttatattataaaattacggctgaaata

SEQ ID NO: 33 - DNA, 38 bp, Artificial Sequence
  synthetic oligonucleotide
aatattcactttttattcttcaaagatttgagctaatt

SEQ ID NO: 34 - DNA, 39 bp, Artificial Sequence
  synthetic oligonucleotide
gaggatatggaaatccatctctactttattgttttcttc

SEQ ID NO: 35 - DNA, 39 bp, Artificial Sequence
  synthetic oligonucleotide
tggaaatccatctctactttattgttttcttcaaatatt

SEQ ID NO: 36 - DNA, 34 bp, Artificial Sequence
  synthetic oligonucleotide
ggtgttggtgaagatataccaagtgattatccat

SEQ ID NO: 37 - DNA, 31 bp, Artificial Sequence
  synthetic oligonucleotide
taataagtgaggtgcgttaatattgccatta

SEQ ID NO: 38 - DNA, 31 bp, Artificial Sequence
  synthetic oligonucleotide
taattaaagcgattgatggtgatacggttaa

SEQ ID NO: 39 - DNA, 31 bp, Artificial Sequence
  synthetic oligonucleotide
tttcgtaaatgcacttgcttcaggaccatat

SEQ ID NO: 40 - DNA, 52 bp, Artificial Sequence
  synthetic oligonucleotide
cgcgctccattaacattatagtcactatttacatctttccattgagagcgcg

SEQ ID NO: 41 - DNA, 37 bp, Artificial Sequence
  synthetic oligonucleotide
cgcagggccgacctgaccaaatgccagcatccctgcg

SEQ ID NO: 42 - DNA, 46 bp, Artificial Sequence
  synthetic oligonucleotide
cgcgcctggacgtattctcaatcactggtcgtggtactgaggcgcg

SEQ ID NO: 43 - DNA, 28 bp, Artificial Sequence
  synthetic oligonucleotide
ggattggaggtcaaataaatgacactgn

SEQ ID NO: 44 - DNA, 34 bp, Artificial Sequence
  synthetic oligonucleotide
tttcaatgtgtttatcaaaaatgcattactatcn

SEQ ID NO: 45 - DNA, 23 bp, Artificial Sequence
  synthetic oligonucleotide
taaagcgggagtcaatgtaacgn

SEQ ID NO: 46 - DNA, 22 bp, Artificial Sequence
  synthetic oligonucleotide
cacagttgcaggcctggttacn

SEQ ID NO: 47 - DNA, 26 bp, Artificial Sequence
  synthetic oligonucleotide
tctgacaaaccattcatgatgccagn

SEQ ID NO: 48 - DNA, 31 bp, Artificial Sequence
  synthetic oligonucleotide
gcttcagcgtagtctaataatttacggaacn

SEQ ID NO: 49 - DNA, 27 bp, Artificial Sequence
  synthetic oligonucleotide
ggattggaggtcaaataaatgacactg

SEQ ID NO: 50 - DNA, 33 bp, Artificial Sequence
  synthetic oligonucleotide
tttcaatgtgtttatcaaaaatgcattactatc

SEQ ID NO: 51 - DNA, 22 bp, Artificial Sequence
  synthetic oligonucleotide
taaagcgggagtcaatgtaacg

SEQ ID NO: 52 - DNA, 21 bp, Artificial Sequence
  synthetic oligonucleotide
cacagttgcaggcctggttac

SEQ ID NO: 53 - DNA, 25 bp, Artificial Sequence
  synthetic oligonucleotide
tctgacaaaccattcatgatgccag

SEQ ID NO: 54 - DNA, 30 bp, Artificial Sequence
  synthetic oligonucleotide
gcttcagcgtagtctaataatttacggaac

(19) United States
(12) Patent Application Publication
(10) Pub. No.: US 2011/0086354 A1
(43) Pub. Date: Apr. 14, 2011

(54) METHODS AND COMPOSITIONS FOR MULTIPLEX PCR AMPLIFICATIONS

(76) Inventors: Tzvi TZUBERY, Ofra (IL); Shira Glasner, Jerusalem (IL); Tal Salomon, D.N. Hefer (IL); Boaz Arieli, Mevasseret Zion (IL); Aryeh Gassel, Jerusalem (IL); Maoz Tal, D.N. Haela (IL); Raphael Gassel, Jerusalem (IL)

(21) Appl. No.: 12/903,401

(22) Filed: Oct. 13, 2010

Related U.S. Application Data
(60) Provisional application No. 61/272,608, filed on Oct. 13, 2009.
(60) Provisional application No. 61/272,833, filed on Nov. 9, 2009.

(51) Int. Cl. C12Q 1/68; C12N 9/00; C12N 9/22

(52) U.S. Cl. 435/6; 435/183; 435/199

(57) ABSTRACT

The present invention concerns in general PCR reaction mixtures comprising a mixture of hot-start primers and non-hot-start primers for a given target sequence, including multiplex PCR reaction mixtures; methods utilizing same for detection of one or more target polynucleotide sequences; and kits comprising same.

### CROSS-REFERENCE TO RELATED APPLICATIONS

This application claims priority from U.S. provisional application 61/272,608, filed on Oct. 13, 2009, and U.S. provisional application 61/272,833, filed on Nov. 9, 2009, each of which is incorporated herein by reference.

### FIELD OF THE INVENTION

The present invention generally concerns polymerase chain reaction (PCR) mixtures comprising a mixture of hot-start primers and non-hot-start primers for a given target sequence, including multiplex PCR reaction mixtures; methods utilizing same for detection of a target polynucleotide sequence of interest; and kits comprising same.

### BACKGROUND OF THE INVENTION

### Polymerase Chain Reaction (PCR) and Real-Time Quantitative PCR

The invention of the polymerase chain reaction (PCR) made possible the in vitro amplification of nucleic acid sequences. PCR is described in U.S. Pat. Nos. 4,683,195; 4,683,202; and 4,965,188.

Additionally, commercial vendors, such as Applied Biosystems (Foster City, Calif.), market PCR reagents and publish PCR protocols.

PCR is typically begun with an initial denaturation step, to enable efficient utilization of template in the first amplification cycle, followed by cycles of PCR amplification. In each cycle of PCR amplification, a double-stranded target sequence is denatured, primers are annealed to each strand of the denatured target, and the primers are extended by the action of a DNA polymerase, referred to as the “denaturation”, “annealing”, and “extension” steps. A final extension step may be included to fill-in the protruding ends of newly synthesized PCR products. The specificity of amplification depends on the specificity of primer hybridization. Primers are selected to be complementary to, or substantially complementary to, sequences occurring at the 3′ end of each strand of the target nucleic acid sequence.

Quantitative PCR, sometimes referred to as “real-time PCR” or “qPCR”, utilizes the same amplification scheme as PCR, with 2 oligonucleotide primers flanking the DNA segment to be amplified. In qPCR, the reaction products are monitored as they are formed. Several methods that rely on fluorescent labeling can be used for real-time monitoring. One common method used in real-time monitoring employs DNA-binding fluorescent dyes such as SYBR® Green fluorescent dye. Another method adds a target-specific oligonucleotide probe that is labeled at 1 end with a fluorescent tag and at the other end with a fluorescent quencher (FRET Probe). Fluorescence resonance energy transfer (FRET) is an energy transfer mechanism between 2 fluorescent molecules. In the TaqMan® variant, the fluorescent label at one end of the oligonucleotide is excited at its specific fluorescence excitation wavelength. This excited state is then non-radioactively transferred to the quencher molecule label at the other end of the oligonucleotide. In a quantitative PCR reaction, the fluorescent labels of those probes that bind to the DNA target are cleaved from the probe during primer extension, thereby releasing the fluorescent tag to emit a signal at its specific fluorescent excitation wavelength without the energy being transferred.

The signal emitted by the oligonucleotide FRET probe typically increases in direct proportion to the amount of PCR product in the reaction. By recording the amount of fluorescence emission at each cycle, the PCR reaction is monitored during the exponential phase when the first significant increase in the amount of PCR product correlates to the initial amount of target template. The higher the starting copy number of the nucleic acid target, the sooner a significant increase in fluorescence is observed. A significant increase in fluorescence above the baseline value measured at each cycle above cycle 10 indicates the detection of accumulated PCR product. Individuals skilled in the art will recognize additional well known methods including Molecular Beacons, described in U.S. Pat. No. 5,925,517, U.S. Pat. No. 6,037,130, U.S. Pat. No. 6,103,476, U.S. Pat. No. 6,150,097, U.S. Pat. No. 6,461,817 and U.S. Pat. No. 7,385,043, Eclipse Probes, and the use of fluorescent labeled primers as in the Scorpion Primer and Sunrise Primer variants.

Use of qPCR to Detect Polynucleotide Sequences of Interest in Clinical Specimens

qPCR has been used to detect target polynucleotide sequences of interest in test samples. One exemplary type of target polynucleotide sequences are those characteristic of pathogens of interest, typically assayed in clinical specimens to test for infectious disease. U.S. Pat. No. 6,664,080 to Klaus Pfeffer, entitled “TaqMan™-PCR for the detection of pathogenic E. coli strains”, describes probes and primers useful in PCR-based assays for detecting subgroups of pathogenic E. coli strains, including enterotoxigenic, enteroaggregative, enteroinvasive, enteropathogenic and enterohemorrhagic E. coli strains. qPCR has also been used to detect viruses, as described inter alia in US Pat. App. No. 2009/0181363, entitled “Non-Invasive Detection of Fish Viruses by Real-Time PCR”, US Pat. App. No. 2006/0177818, entitled “Method of detection of classical swine fever”, and references cited therein.

qPCR has also been used to specifically detect antibiotic-resistant pathogens such as Methicillin-resistant Staphylococcus aureus (MRSA). US Pat. App. No. 2009/0081663 to Yosef Paitan, entitled “Methods, Compositions and Kits for Detection and Analysis of Antibiotic-Resistant Bacteria”, for example, describes use of multiplex qPCR that amplifies (a) a gene specific for the target bacteria; (b) an antibiotic-resistance gene; and (c) the bridging region between a known region of the bacterial genome and the usual site of integration of the antibiotic-resistance cassette. U.S. Pat. No. 6,329,138 to Hans De Beenhouwer et al, entitled “Method for detection of the antibiotic resistance spectrum of mycobacterium species”, describes probes and primers useful in PCR-based assays for detecting strains of M. tuberculosis resistant to rifampicin and analogues thereof U.S. Pat. No. 7,045,291 to Nancy Hanson et al, entitled “Multiplex PCR for the detection of AmpC beta-lactamase genes”, describes probes for detecting certain AmpC beta-lactamases. U.S. Pat. No. 5,994,066, U.S. Pat. No. 6,001,564, and International Pat. App. No. WO/1996/008582 to Bergeron et al, assigned to Creighton University, and describe probes for detecting a bacterial antibiotic resistance gene selected from of blaₜₑₘ, bla_{rod}, blaₛₕᵥ, blaₒₓₐ, bal_{z}, aadB, aacC1, aacC2, aacC3, aacA4, aac6′-IIa, ermA, ermB, ermC, mecA, vanes, vanX, vanB, satA, aacA-aphD, vat, vga, msrA, sul and int from a specific bacterial species selected from H. influenzae, K. pneumoniae, P. mirabilis, S. saprophyticus, E. faecium, and various Streptococcus species. US Pat. App. No. 2004/0185478 describes species-specific, genus-specific and universal DNA probes and amplification primers for rapidly detection and identification of bacterial and fungal pathogens and their antibiotic resistance genes from clinical specimens for diagnosis in microbiology laboratories. See also Huletsky A et al (New real-time PCR assay for rapid detection of methicillin-resistant S. aureus directly from specimens containing a mixture of staphylococci. J Clin Microbiol. 2004; 42(5):1875-84).

qPCR has also been used to specifically detect target polynucleotide sequences in forensic samples, as described inter alia in U.S. Pat. Nos. 6,673,541 and 5,861,504 and US patent application nos. 2009/0023603, 2006/0099620, and 2008/0286773. Often, several amplification reactions are run in a multiplex assay to make best use of very limited available DNA sample.

qPCR has also been used to specifically detect human DNA markers, for example BRCA1 and BRCA2, as described inter alia in U.S. Pat. Nos. 7,507,800 and 6,083,698 and in US patent application no. 2005/0095592. Often, several amplification reactions are run in a multiplex assay to minimize cost and rapidity.

### Non-Specific Hybridization in PCR

In PCR, generation of non-specific amplification products may occur. Artifacts are sometimes derived from inappropriate hybridization products designated as “primer-dimers” (Rychlik, 1995) and “primer-probe dimers”, resulting from weak interactions between primers or between primers and probes such as TaqMan® and Molecular Beacon probes. Undesired products may form as a result of weak complementarity between the 3′ ends of a primer and bases in non-target oligonucleotide strands in the reaction mix. This enables annealing of the primer to the non-target strand, followed by initiation and elongation of the non-specific dimer by the thermo-stable DNA polymerase, leading to amplification of undesired byproducts.

In real-time PCR, this problem may be exacerbated due to the sensitivity of the assay. This problem is further exacerbated in multiplex PCR amplification reactions due to the presence of several primer sets and probes in the reaction mix, presenting increased likelihood of non-specific hybridization. These problems may lead to reduced sensitivity and data quality, as discussed further in Chou et al, 1992, Nucleic Acids Research 20(7):1717-1723. Non-specific amplification also reduces the number of cycles that can be run, decreasing the sensitivity of the assay.

### “Hot-Start” Methods and Other Solutions to Non-Specific Amplification

Non-specific amplification can be reduced by reducing the formation of extension products from primers bound to non-target sequences prior to the start of the reaction. One method of addressing the problem of non-specific amplification is referred to as a “hot-start” protocol, wherein one or more critical reagents are withheld from the reaction mixture until the temperature is raised sufficiently to provide the necessary hybridization specificity. In this manner, the reaction mixture cannot support primer extension during the time that the reaction conditions allow non-specific primer hybridization.

Hot-start methods can be carried out manually by opening the reaction tube after the initial high temperature incubation step and adding the missing reagents. However, it will be appreciated that manual hot-start methods are labor intensive, and there is also increased risk of contamination of the reaction mixture. Hot-start methods that use a heat labile material, such as wax, to separate or sequester reaction components are described in U.S. Pat. No. 5,411,876 and Chou et al., 1992, supra. In these methods, a high temperature pre-reaction incubation melts the heat labile material, thereby allowing the reagents to mix.

Another method of reducing the formation of extension products from primers bound to non-target sequences prior to the start of the reaction relies on inhibition of the DNA polymerase using a compound that non-covalently binds to the DNA polymerase in a heat-reversible manner. U.S. Pat. No. 5,338,671 describes the use of antibodies specific for a thermostable DNA polymerase to inhibit the DNA polymerase activity. A disadvantage of this method is that the production of antibodies specific to the DNA polymerase is expensive and time-consuming, especially in large quantities. Furthermore, the addition of antibodies to a reaction mixture may require redesign of the amplification reaction.

U.S. Pat. Nos. 5,677,152 and 5,773,258 to David Edward Birch, et al, assigned to Roche Molecular Systems, Inc, both entitled “Nucleic Acid Amplification Using a Reversible Inactivated Thermostable Enzyme”, describe another method of reducing non-specific amplification reactions, known as “hot-start polymerase”. A Taq polymerase enzyme is reversibly inactivated by chemical modification. Enzyme activity is recovered by incubating the reaction mixture at an elevated temperature prior to, or as part of, the amplification reaction. Non-specific amplification is reduced because the reaction mixture does not support the formation of extension products prior to the activating high-temperature incubation.

US Pat. App, No. 2004/0197800 to Michael Borns, assigned to Stratagene, discloses blends of hot-start and non-hot-start DNA polymerases.

Another method of reducing non-specific amplification reactions is described in US Pat. App. Nos. 2005/0255486 and 2008/0038724 to Mark Behlke et al, assigned to Integrated DNA Technologies, Inc, entitled “Methods for amplifying polymeric nucleic acids”. The primer set used in the amplification is composed of a mixture of two types of primers. For extension in one direction, the primers, while functional as primers, all contain modifications that abrogate their ability to serve as templates that can be copied by DNA polymerases. For extension in the opposite direction, the set includes at least one primer that can serve as a template and be replicated by DNA polymerases throughout its length. At least one primer in the primer set hybridizes to the nucleic acid polymer. It is preferred that the non-replicable primer hybridizes to the nucleic acid polymer and is extended to produce an extension product that contains sequence from the nucleic acid polymer to which the replicable primer then hybridizes.

An article by LG Puskás et al (Reduction of mispriming in amplification reactions with restricted PCR; Genome Res. 1995; 5(3):309-11) describes another strategy for reducing non-specific amplification, by adding 3′-dideoxy-terminated competitor oligonucleotides to the amplification mixture. The competitors nucleotides reportedly inhibit nonspecific amplification reactions.

### Primers Modified to Reduce Non-Specific Hybridization

U.S. Pat. No. 6,001,611 to Stephen Gordon Will, assigned to Roche Molecular Systems, Inc, entitled “Modified nucleic acid amplification primers”, describes another type of modified oligonucleotide primers, having the general structure:

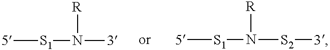

wherein S₁ represents a first sequence of nucleotides between about 5 and about 50 nucleotides in length;
wherein S₂ represents a second sequence between one and three nucleotides in length;
wherein N represents a nucleotide that contains a purine or pyrimidine base that contains an exocyclic amine;
wherein R represents a modifier group, wherein R is covalently bound to the nitrogen atom of the exocyclic amine, and wherein R has the structure:

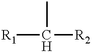

The modified primers reportedly reduce non-specific amplification, especially primer-dimer formation, and/or concomitantly increase the yield of the intended target relative to an amplification carried out with unmodified primers, by interfering with Watson-Crick base pairing of the modified base with the complementary base and/or extension of the modified primer.

### Hot-Start Primers

Another mechanism for reducing non-specific reactions is the use of primers that are activated at elevated temperatures, known as “hot-start primers”. One type of hot-start primers is described in U.S. Pat. No. 6,482,590 to Edwin Ullman et al, assigned to Aventis Behring GmbH, entitled “Method for polynucleotide amplification”, which describes modified oligonucleotides having a 3′-end that is inefficiently extendable along any polynucleotide. The modified nucleotides are reportedly relatively resistant to the 3′-exonuclease activity of Pfu polymerase activity at ambient temperatures, but undergo slow degradation to remove the modified nucleotides as the temperature is increased, resulting in gradual introduction of functional primers into the PCR reaction, thereby improving overall specificity.

US Pat. App. No. 2007/0128621, assigned to Applera Corporation, describes PCR reaction mixtures for multiplex amplification of mRNA and micro RNA targets, containing a hot-start primer having a stem-loop structure and directed against the mRNA target and a regular primer directed against the micro RNA target.

US Pat. App. No. 2007/0281308 to Gerald Zon et al, entitled “Chemically Modified Oligonucleotide Primers for Nucleic Acid Amplification”, discloses primers containing a heat-removable modification group, preferably at the 3′ terminus, which dissociates during the initial denaturation step of the amplification. The inactive primers may be present in a mixed population with functional primers.

International patent application WO 2009/004630 to Ofer Peleg of Genaphora Ltd, entitled “Chimeric Primers for Improved Nucleic Acid Amplification Reactions”, and an article by Peleg et al (The use of chimeric DNA/RNA primers in quantitative PCR for the detection of Ehrlichia canis and Babesia canis. Appl Environ Microbiol. 2009; 75(19):6393-8) describe another type of primer used for reducing non-specific amplification reactions. These primers, which incorporate a few ribonucleotides in non-adjacent positions in proximity to the initiation zone, reportedly decrease the formation of non-specific amplification products.

Another type of hot-start primers is described in articles by M Ailenberg et al. (Controlled hot start and improved specificity in carrying out PCR utilizing touch-up and loop incorporated primers (TULIPS). Biotechniques. 2000; 29(5):1018-20, 1022-4) and O K Kaboev et al (PCR hot start using primers with the structure of molecular beacons (hairpin-like structure). Nucleic Acids Res. 2000; 28(21):E94), which describe loop primers that contain additional non-template 5′ sequence that self-anneals to the 3′ region and inhibits initiation of polymerization. Upon heating of the reaction mixture, the loop regions of the primers reportedly melt and are activated.

Hot-start primers containing covalent chemical modifications are also described in the literature, US Pat. App. No. 2003/0119150 to Waltraud Ankenbauer et al, assigned to Roche Diagnostics, entitled “Composition and method for hot start nucleic acid amplification”, describes use of primers containing chemical modifications at the 3′ end of at least one primer. The reaction mixture also includes a thermostable exonuclease that is inactive at ambient temperatures, thus leaving the modified primer unaffected. When the temperature is increased, the exonuclease becomes active and removes the 3′ modification of the primer, activating the primer for amplification.

US Pat. App. No. 2003/0162199 to Alex Bonner, assigned to BioLink Partners, Inc, entitled “Reversible chemical modification of nucleic acids and improved method for nucleic acid hybridization” describes modification of target nucleic acid(s), primer(s) or nucleoside triphosphates with a removable protecting group that is releasable from the nucleic acids using heat. The chemical modification can be selected from glyoxal, derivatives thereof, 3,4,5,6-tetrahydrophthalic anhydride, 3-ethoxy-2-ketobutyraldehyde(kethoxal), ninhydrin, hydroxyacetone, diethyl oxalate, diethyl mesoxalate, 1,2-naphthoquinone-4-sulfonic acid, pyruvaldehyde, amides, γ-carboxyacylamides, amidines, and carbamates.

An article by A V Lebedev et al. (Hot Start PCR with heat-activatable primers: a novel approach for improved PCR performance. Nucleic Acids Res. 2008. 36(20):e131) describes another type of hot start primers that contain 1-2 thermolabile, 4-oxo-1-pentyl (OXP) phosphotriester (PTE) modification groups at 3′-terminal and 3′-penultimate inter-nucleotide linkages. These modifications reportedly impair DNA polymerase primer extension under pre-reaction conditions. Incubation of the OXP-modified primers at elevated temperatures yields the corresponding unmodified phosphodiester (PDE) primer, which is a suitable DNA polymerase substrate.

U.S. Pat. No. 6,794,142, to Walter J. Laird et al, assigned to Roche Molecular Systems, Inc, entitled “Amplification using modified primers”, describes hot-start primers containing a modified nucleotide within the three 3′ terminal nucleotide positions; wherein the modified nucleotide is a 2′-O-methyl nucleotide, 2′-fluoro-nucleotide, 2′-amino nucleotide, or arabinose nucleotide. These modified primers reportedly reduce non-specific amplification by increasing the time required for the initial primer extension to occur, probably by rendering the primer-target duplex a less preferred template for extension. This reduces the likelihood that an unstable, transient hybridization duplex, such as between primers under pre-reaction conditions, will exist for a sufficient time to permit primer extension.

A different type of hot-start primers is described in an article by D D Young et al (Light-triggered polymerase chain reaction. Chem Commun (Camb). 2008; (4):462-4). These primers are modified with a sterically demanding caging group that is removable by UV irradiation. The unmodified primers reportedly fail to catalyze a PCR reaction until exposed to UV irradiation, after which the reaction proceeds normally. Such primers are suitable for a hot-start protocol wherein the reaction mixture is heated to the annealing temperature, then exposed to UV irradiation.

### “Ribo-Primers”

US Pat. App. Nos. and 20090325169 and 20100167353, both assigned to Integrated DNA Technologies Inc. (IDT) and entitled “RNase H-Based Assays Utilizing Modified RNA Monomers”, describe another type of hot-start PCR primers, “ribo-primers”. These primers are suitable for PCR reaction mixes wherein the hot start component is a thermostable RNase H or other nicking enzyme that gains activity at the elevated temperatures employed in the reaction. The modified primers have an internal modified nucleotide that generates an RNase H2 cleavage site when bound to DNA. In addition, the primers contain a 3′ blocking group, which precludes the ability of the primers to support PCR until the blocking group is removed. Cleavage by RNase H2 requires duplex formation of primer and target and increases the specificity of priming, which reduces the impact of primer-dimer formation, thereby lowering the background signal and improving the overall reaction specificity.

### Other Types of Non-Functional Primers in Nucleic Acid Amplification Reactions

A number of other references describe use of non-functional or antagonistic primers in nucleic acid amplification reactions. US Pat. App. No. 2003/0104430 and International Application WO00/61817 to Michael Nerenberg et al, entitled “Amplification and separation of nucleic acid sequences using strand displacement amplification and bioelectronic microchip technology”, for example, describe use of non-cleavable primers in a primer mix for strand displacement amplification (SDA), in combination with bioelectronic microchip technology. SDA is an isothermal and asynchronous nucleic acid amplification process. The non-cleavable primers are intended to retain signal that was been nicked prior to denaturation of the double-stranded template, thus improving signal intensity in anchored SDA, or to bias amplification towards a desired direction. The non-cleavable primers may be provided in combination with normal SDA primers.

U.S. Pat. No. 5,712,386 to Chang-Ning Wang et al, assigned to Biotronics Corporation, discloses blocking nucleotides that hybridize to primers. The blocking nucleotides and primers may be present in a molar ratio of blocking nucleotide/primer of between 0.3-5.0.

### Problem of Failure to Amplify in Multiplex PCR Reactions Using Hot-Start Primers

Ideally, to minimize cost and complexity and to obtain test results as rapidly as possible, qPCR assays should not require extensive DNA purification procedures and should be performed in a single test tube. However, certain types of multiplex PCR, for example (a) detection of antibiotic-resistant pathogens; (b) multiplex PCR screening tests for detection of one or more of a panel of bacterial or viral pathogens or human DNA markers; and (c) PCR assays of forensic samples, present a unique challenge in this regard. Since several amplification reactions must be conducted in parallel, there is increased possibility of non-specific amplifications, which are liable to produce a significant signal in such highly-sensitive assays. Sequence variability among bacterial strains adds an additional level of complexity to the challenge. Accordingly, there is a pressing need in the art for reaction mixtures that enable multiplex PCR amplification of clinical specimens. Hot-start primers such as ribo-primers have been developed to address these challenges.

As demonstrated herein, however, an additional problem may be observed with hot-start primers such as ribo-primers. These primers may fail to efficiently amplify one or more targets in DNA samples that have not been extensively purified, particularly in multiplex reactions. Thus, the prior art has been unable to effectively address the problem of providing reaction mixtures that enable multiplex PCR amplification of clinical specimens. Methods and compositions of the present invention are intended to overcome these deficiencies of the prior art.

### SUMMARY OF THE INVENTION

The invention described hereinbelow is directed to overcoming the limitations of existing qPCR methods, particularly at least some of the deficiencies described in the Background section.

Embodiments of the present invention are directed to methods and reagents for primer-based in vitro nucleic acid amplifications, which provide a simple and economical solution to the problem of efficient multiplex PCR amplification.

One aspect of the invention relates to kits for the in vitro amplification of a nucleic acid sequence using a primer-based amplification reaction, wherein the kits comprise a mixture of a hot-start primer and a regular primer that are both directed to the same end of a particular amplicon. The sequences recognized by the different primers may be identical, overlapping, or non-overlapping. A kit typically will comprise one or more amplification reagents, e.g., a nucleic acid polymerase, nucleoside triphosphates, and/or suitable buffers. Further, a kit may comprise additional components, such as a means for detecting the amplified product.

Another aspect of the present invention relates to methods for amplifying a nucleic acid, the methods including the step of conducting a primer-based nucleic acid amplification reaction using a mixture of a hot-start primer and a regular primer that are both directed to the same end of a particular amplicon. Thus, the present invention provides a method for amplifying a target nucleic acid contained in a sample, comprising the steps of: (a) providing an amplification reaction mixture comprising the target nucleic acid and a pair of primers, comprising said mixture of a hot-start primer and a regular primer; and (b) treating the reaction mixture of step (a) under conditions suitable for the amplification of the nucleic acid. In certain embodiments, multiple primer sets are included such that a multiplex PCR reaction is performed. In certain other embodiments, the sample being analyzed is from a clinical specimen and the nucleic acids of the sample have not been extensively purified.

In certain embodiments of the invention, methods and compositions of the present invention utilize both primers directed against one or more targets, which primers are mixtures of hot-start and regular primers, and primers directed against one or more other targets which primers are hot-start primers only.

Another aspect of the invention relates to amplification reaction mixtures that comprise a mixture of a hot-start primer and a regular primer that are both directed to the same end of a particular amplicon.

Another aspect of the invention relates to improved methods for PCR amplification, utilizing at least one hot-start primer that contains an inactivating chemical modification, wherein the inactivating chemical modification is reversible when the hot-start primer is hybridized to a complementary sequence at elevated temperatures, the improvement consisting of provision of a non-hot-start/functional primer directed against the same target (directed against the same amplicon, in the same orientation) as the hot-start primer. In another embodiment, the improvement comprises provision of a non-hot-start/functional primer directed against the same target as the hot-start primer. In certain embodiments, multiple primer sets are included such that a multiplex PCR reaction is performed. In certain other embodiments, the sample being analyzed is from a clinical specimen and the nucleic acids of the sample have not been extensively purified. In some embodiments, the improvement may further comprise additional aspects of the present invention as described herein.

Certain embodiments of the methods and compositions of the present invention enable reduced cost and complexity and the rapid obtaining of test results, particularly since they generally do not require extensive DNA purification procedures and can be performed in a single test tube. In multiplex PCR for screening of forensic samples for detection of multiple human DNA markers that can be collectively used to determine human identity, several amplification reactions are run in parallel to make best use of often very limited available DNA sample. In multiplex PCR for detection of antibiotic-resistant pathogens, several amplification reactions must be conducted in parallel (see, e.g., US Pat. App. No. 2009/0081663), increasing the possibility of non-specific amplifications, which are liable to be detected using highly-sensitive methodology such as qPCR. The same demands exist for multiplex PCR screening tests for detection of one or more of a panel of bacterial or viral pathogens, where the relative quantitative bacterial or viral load is of clinical relevance. In addition, the findings presented herein show that clinical specimens often contain substances that decrease the efficiency of PCR and can inhibit PCR using hot-start primers when mismatches or other factors contributing to a weakening of the primer-target hybridization bonds are present. These challenges are addressed by specific methods and compositions within the scope of the present invention.

Hot-start primers, for example ribo-primers and similar types of primers, reduce primer-dimer formation and other non-specific reactions, thus representing a significant advance in multiplex PCR reactions. However, it has been unexpectedly discovered herein that clinical specimens not subjected to a high degree of purification contain one or more factors that may inhibit PCR reactions utilizing ribo-primers. In some cases, the inhibition may reduce one or more specific amplification signals, negatively impacting assay sensitivity. Certain embodiments of methods and compositions of the present invention unexpectedly were found to dramatically improve this type of inhibition. Even more unexpected was the ability of these methods and compositions to overcome said inhibition without compromising the specificity of the reaction or introducing non-specific amplification products.

As used herein “comprising” is to be interpreted as specifying the presence of the stated features, integers, steps, or components as referred to, but does not preclude the presence or addition of one or more features, integers, steps, or components, or groups thereof. Thus, for example, a primer comprising a given sequence may contain additional nucleic acids at one or both of the 5′ or 3′ ends of that given sequence. Additionally, the term “comprising” is intended to include embodiments encompassed by the terms “consisting essentially of” and “consisting of.” Similarly, the term “consisting essentially of” is intended to include embodiments encompassed by the term “consisting of.”

When an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

### BRIEF DESCRIPTION OF THE DRAWINGS

FIG. 1. PCR amplification utilizing all ribo-primers and targeting purified DNA from bacterial samples. In this and all subsequent figures, the horizontal axis depicts the cycle number, and the vertical axis depicts the level of fluorescence of the appropriate wavelength.

FIG. 2A. PCR amplification utilizing all hot-start primers and targeting DNA extracted from MRSA Strain #2 in two versions: Directly extracted bacteria (“Direct samples”) and bacterial spiked onto nasal swab samples (“Spiked Nasal samples”). B. PCR amplification utilizing all hot-start primers and targeting DNA extracted from MRSA Strain #1 in two versions: Direct samples and Spiked Nasal samples.

FIG. 3A. PCR amplification targeting DNA extracted from MRSA Strain #3 derived from Spiked Nasal samples utilizing two alternative primer mixtures. Mixture #1 contains all hot-start primers, and Mixture #2 contains hot-start primers plus a set of non-hot-start primers at 10% of the hot-start primer molar concentration for the three clinical gene targeting assays, with hot-start primers only for IC. B. PCR amplification targeting DNA extracted from MRSA Strain #3 derived from Spiked Nasal samples utilizing a third alternative primer mixture, containing the hot-start primers for the internal control targeting assay and regular primers only at 10% concentration for the three clinical gene targeting assays.

FIG. 4A. PCR amplification targeting Direct and Spiked Nasal DNA samples of MRSA Strain #3 and containing hot-start primers only for the OrfX and Internal Control assays and regular primers only for mecA and nuc. B. PCR amplification utilizing all hot-start primers and targeting Direct and Spiked Nasal DNA samples of MRSA Strain #3. C. Side-by-side depiction of amplifications of the Spiked Nasal sample using the PCR mixtures described for A and B.

FIG. 5. PCR amplification targeting unpurified MRSA Strain #3 DNA derived from Spiked Nasal samples. The multiplex assay utilizes hot-start primers only for IC and three different mixtures of primers for orfX, nuc and mecA. Mixture #1: 80% hot-start primers and 20% regular primers. Mixture #2: Regular primers only, in the same amount as Mixture #1. Mixture #3: 100% hot-start primers and 0% regular primers.

FIG. 6. PCR amplification similar to that described for FIG. 5, but with an optimized ratio of regular primers and hot-start primers for the assays targeting the nuc and mecA genes.

### DETAILED DESCRIPTION OF THE INVENTION

The present invention provides PCR reaction mixtures comprising a mixture of hot-start primers and non-hot-start primers directed to the same end of a particular amplicon, including multiplex PCR reaction mixtures; methods utilizing same; and kits comprising same.

In jurisdictions allowing it, all patents, patent applications, and publications mentioned herein, both supra and infra, are incorporated herein by reference.

### General Definition

The term “strain” as used herein, refers to a subset of a bacterial species differing from other bacteria of the same species by an identifiable difference.

In one embodiment, the present invention provides a PCR reaction mixture comprising: a) a set of primers, comprising at least one forward primer and at least one reverse primer, that is capable of amplifying a target polynucleotide sequence characteristic of a species of interest (hereinafter referred to as a “species-specific gene”); and b) an activating enzyme, wherein at least one of said forward primer and said reverse primer is provided as a mixture of hot-start primers and non-hot-start primers. Typically, the reaction mixture further comprises one or more of a DNA polymerase enzyme, deoxynucleoside triphosphates (dNTPs), and a divalent cation, most often a magnesium ion. The hot-start primers contain an inactivating chemical modification that is reversed by the action of the activating enzyme present in the reaction mixture. Each individual hot-start primer molecule becomes a substrate for the activating enzyme when the hot-start primer is hybridized to a sequence complementary to the primer at elevated temperatures. In certain embodiments, the PCR reaction mixture further comprises a probe suitable for real-time PCR.

In some embodiments, the hot-start primers and non-hot-start primers are present in a ratio of hot-start primers to non-hot-start primers of between 1:9 and 19:1, inclusive. The determination of an optimal preferred ratio will vary for different multiplex PCR assays and will depend, in part, upon the likelihood of non-specific binding of the primer. This will in turn depend, in large part, upon the extent of complementarity between one or both of the primers of the assay and themselves, other oligonucleotides, and/or non-target nucleic acid sequences present in the sample. In the presence of high non-specific complementarity, the proportion of non-hot-start primers would typically be limited to 20% or less, in other words a ratio of hot-start to non-hot-start primers of between 4:1 and 19:1, inclusive.

In certain other embodiments, wherein primer hybridization is problematic at only one end of an amplification, a mixture of non-hot-start primers and hot-start primers is use for only one end of the amplification while the other end of the amplification is driven by hot-start primers only. It will be understood to one skilled in the art that empirical methods may be used to determine whether to select the forward primer, the reverse primer, or both for use of a mixture of hot-start and non-hot-start primers.

In another embodiment, the present invention provides a PCR reaction mixture for multiplex PCR, comprising: (a) a first set of primers for amplifying a first target polynucleotide sequence; (b) a second set of primers for amplifying a second target polynucleotide sequence; and (c) an activating enzyme, wherein:
(i) the primers that hybridize to at least one end of the first target polynucleotide sequence are a mixture of hot-start primers and non-hot-start primers, wherein the reaction product of amplifying the first target polynucleotide sequence using the non-hot-start primers is a substrate for amplification using the hot-start primers; and
(ii) the second set of primers consists of hot-start primers.

Typically, the reaction mixture further comprises one or more of a DNA polymerase enzyme, deoxynucleoside triphosphates (dNTPs), and a divalent cation selected from a magnesium, manganese, nickel, or cobalt ion, Those of skill in the art will readily understand that, when reference herein to “the reaction product of amplifying [a target polynucleotide sequence] using the non-hot-start primers”, or similar language, refers to an amplification using forward and reverse non-hot-start primers, if they are present in the reaction mixture. If non-hot-start primers are present on only one end of the amplification, reference is intended to amplification using the non-hot-start primers in combination with the supplied hot-start primers having the reverse orientation. Similarly “amplification using the hot-start primers”, or similar language, refers to an amplification using forward and reverse hot-start primers, if they are present in the reaction mixture. If hot-start primers are present on only one end of the amplification, reference is intended to amplification using the hot-start primers in combination with the supplied non-hot-start primers having the reverse orientation.

In certain embodiments, the hot-start primers contain an inactivating chemical modification that is reversed by the action of the activating enzyme present in the reaction mixture. Individual hot-start primers become a substrate for the activating enzyme when the hot-start primer is hybridized to a sequence complementary to the primer at elevated temperatures. Typically the mixture of hot-start primers and non-hot-start primers is present as a ratio of hot-start primers to non-hot-start primers of between 1:9 and 19:1, inclusive

In certain embodiments, the PCR reaction mixture further comprises a probe suitable for real-time PCR.

As noted above, the determination of an optimal preferred ratio will vary for different multiplex PCR assays and will be dependent, in part, upon the extent of complementarity present in the target-containing reaction mixture that could lead to non-specific hybridization and the extent of inhibition of amplification. In certain embodiments, wherein only 1 of the 2 ends of the amplification appears to be inhibited from extension and where the presence of sequences complementary to the hot-start primers of the inhibited assay is low so that the use of non-hot-start primers for that inhibited assay does not substantively increase the presence of non-specific amplification, the mixture of hot-start-primers and non-hot-start primers in that position will optimally include a majority of non-hot-start primers. It is shown herein that even in such cases, the hot-start primers advantageously slow amplification mediated by the non-hot-start primers, possibly by competing with them for hybridization to target sequences.

Prior to the experiments presented herein, multiplex PCR using ribo-primers had been successfully performed on DNA samples that had been purified using the DNeasy™ Blood & Tissue Kit (QIAGEN GmbH). It was then decided to determine whether these assays would function well in a clinical setting, for example isolated nucleic acids from nasal swabs without using extensive purification procedures. To this end, purified DNA was spiked onto nasal swabs of volunteers. As described hereinbelow, it was found that biological factors were present in the nasal swabs that inhibited multiplex PCR using ribo-primers. The inhibition could be overcome by either subjecting the DNA to more extensive purification, increasing the amount of template, or performing the assays individually in single-plex assays. Each of these solutions was impractical and/or undesirable in a clinical setting, and the present invention was developed to address this deficiency.

Kits of some embodiments of the present invention are capable of being successfully used with real-world samples, without a requirement for extensive nucleic acid purification, which itself leads to a number of practical advantages. For example, the Detect-Ready™ MRSA Lysis Kit (Molecular Detection, Inc.) may be used, as described in Example 2. The ability of kits of some embodiments of the present invention to simplify sample preparation prior to PCR amplification thus can save time and expense for health-care facilities. The saved time can even, in some embodiments, mean the difference between a timely clinical intervention and one that is too late to save the patient. In other embodiments, simplifying sample preparation also reduces the number of opportunities for sample contamination.

As provided herein in the Examples, hot-start primer qPCR amplification of target polynucleotide sequences containing a primer-target mismatch is noticeably less robust (Example 1). This inhibition can also be observed in multiplex PCR performed on nucleic acids derived from clinical specimens that have not been subjected to extensive purification (Example 2). “Derived from” in this context is intended to encompass any form of homogenization, dissolution in a solvent or solution, fractionation, or a combination thereof, alone or in combination with any other appropriate means by which a sample suitable for PCR amplification can be generated from a clinical specimen. Certain embodiments of methods and compositions of the present invention unexpectedly were found to dramatically improve the inhibition observed with ribo-primers in certain PCT reactions.

In some embodiments, methods and compositions of the present invention are indicated for nucleic acid preparations derived from clinical specimens that have not necessarily been subjected to extensive purification. For example, in a non-limiting embodiment, methods and compositions of the present invention are suitable for use with samples having an A₂₆₀/A₂₈₀ ratio of less than 2.0 (the A₂₆₀/A₂₈₀ ratio is a measure of the purity of a nucleic acid preparation). In another embodiment, methods and compositions of the present invention are suitable for use with samples having an A₂₆₀/A₂₈₀ ratio of less than 1.9. In another embodiment, methods and compositions of the present invention are suitable for use with samples having an A₂₆₀/A₂₈₀ ratio of less than 1.8. In another embodiment, methods and compositions of the present invention are suitable for use with samples having an A₂₆₀/A₂₈₀ ratio of less than 1.7. In another embodiment, methods and compositions of the present invention are suitable for use with samples having an A₂₆₀/A₂₈₀ ratio that is between 1.0-2.0. In another embodiment, methods and compositions of the present invention are suitable for use with samples having an A₂₆₀/A₂₈₀ ratio that is between 1.0-1.9. In another embodiment, methods and compositions of the present invention are suitable for use with samples having an A₂₆₀/A₂₈₀ ratio that is between 1.0-1.8. In another embodiment, methods and compositions of the present invention are suitable for use with samples having an A₂₆₀/A₂₈₀ ratio that is between 1.0-1.7. Each possibility may be considered as being a separate embodiment of the present invention.

An additional potential pitfall of multiplex PCR on clinical specimens is shown in Example 4, which shows that amplifications utilizing only hot-start primers are likely to be inhibited in the presence of other amplifications that utilize only regular primers. When performed individually using the same specimens, the amplifications were not inhibited (data not shown). Without wishing to be bound by any particular theory or mechanism of action, the inhibition of the hot-start amplifications in this case is believed to be due to competition between amplifications for resources such as dNTPs in the reaction tube.

As further provided herein, use of a mixture of hot-start and regular primers for a single amplification, whether the regular and hot-start primers recognize the same target sequence (Examples 5-6) or are designed in a nested configuration wherein the regular primers flank the hot-start primers (Example 3), overcomes the limitations of using ribo-primers in multiplex qPCR, enabling efficient amplification of all targets.

Furthermore, some embodiments of the present invention increase the number of amplicons that can be amplified from unpurified nucleic acids from clinical specimen, relative to the prior art solutions known to the inventors, thereby significantly lowering cost and increasing assay rapidity.

Also provided herein are reaction mixtures including a set of primers for amplifying a particular target polynucleotide sequence, wherein the primers for amplifying at least one end of the target sequence include a mixture of 10-95% of hot-start primers and 5-90% of non-hot-start primers. As provided herein, such reaction mixtures exhibit properties superior to mixtures containing either hot-start primers alone or non-hot-start primers alone.

For example, the forward primer set for amplifying the target sequence may be composed of a mixture of hot-start and non-hot-start primers. In another embodiment, the reverse primer set is composed of a mixture of hot-start and non-hot-start primers. In other embodiments, both the forward and reverse primer sets are composed of a mixture of hot-start and non-hot-start primers. Each possibility may be considered as being a separate embodiment of the present invention.

In another embodiment, as exemplified herein for the reverse primers of a bacterial “bridging region”, sequence variability may necessitate several forward primers and/or several reverse primers for amplification of sequence variants a given target (“alternate primers”). In this case, in certain embodiments, each set of primers of the alternate primers (a set of primers is used in this case to denote primers recognizing a particular sequence variant) is a mixture of hot-start and non-hot-start primers in a given ratio. In certain other embodiments, one or more of the sets of alternate primers consists of hot-start primers, while the other set of alternate primers contains a mixture of hot-start and non-hot-start primers in a given ratio. Typically, the hot-start:non-hot-start mixture ratio is the same for each set of the alternate primers. Each possibility may be considered as being a separate embodiment of the present invention.

As provided herein, reaction mixtures of the present invention are particularly efficacious wherein the amplification product using the non-hot-start primers is a substrate for amplification using the hot-start primers. In some embodiments, as illustrated in the Examples herein, the complementary sequences of the non-hot-start primers may flank the complementary sequences of the hot-start primers (a “nested configuration”). In other embodiments, if non-hot-start primers are only present in one orientation, the non-hot-start primers may bind a sequence upstream of the corresponding hot-start primers. In other embodiments, as illustrated in the Examples herein, the complementary sequences of the non-hot-start primers may overlap the complementary sequences of the hot-start primers, or may be identical to the complementary sequences of the hot-start primers. In some cases, the hot-start primers may be identical to the non-hot-start primers except for the hot-start modification. For example, CGCATGACCCAAGGGCArAAGCG-▪ (SEQ ID NO: 5) is identical to the sequence CGCATGACCCAAGGGCAAAGCG (SEQ ID NO: 35), except for the presence of the ribonucleoside residue (“ribo-base”) and blocking group in the former. In other embodiments, the hot-start primers may be identical to the non-hot-start primers except for the hot-start modification and additional complementary sequence 3′ to the ribo-base, as exemplified herein inter alia by sequence pairs 5 and 18 and 12 and 25, respectively. In light of the findings presented herein, it will be understood by those skilled in the art that the efficacy of methods and compositions of the present invention does not depend on the exact relationship of the target sequences of the non-hot-start and hot-start primers, provided that the amplification product of the non-hot-start primers is a substrate for amplification using the hot-start primers. Each possibility may be considered as being a separate embodiment of the present invention.

In another embodiment, in addition to a first set of primers, the PCR reaction mixture comprises a second set of primers targeting a different target polynucleotide sequence. It will be apparent to those skilled in the art that the second target polynucleotide sequence may be any target sequence known in the art.

In some non-limiting embodiments, another mixture of hot-start primers and non-hot-start primers directed against a different polynucleotide sequence may be utilized, also typically present in a ratio of hot-start primers to non-hot-start primers of between 1:9 and 19:1. In other embodiments, any of the other ratios provided herein may be used, each of which constitutes a separate embodiment of the present invention. As provided herein in Examples 3 and 5, multiplex amplification reactions containing primer sets that are exclusively hot-start primers and containing other primers sets that are mixtures of hot-start and non-hot-start primers, with each primer set targeting a different nucleic acid target, are shown to be particularly efficacious in multiplex PCR on clinical specimens, as exemplified by assays detecting antibiotic-resistant pathogen strains in clinical specimens that may contain inhibitors of hot-start primer PCR.

For example, the forward primers for amplifying the second target polynucleotide sequence may be composed of a mixture of hot-start and non-hot-start primers. In another embodiment, the reverse primers are composed of a mixture of hot-start and non-hot-start primers. In other embodiments, both the forward and reverse primers are composed of a mixture of hot-start and non-hot-start primers. Each possibility may be considered as being a separate embodiment of the present invention.

In another embodiment, in addition to the first and second sets of primers, the PCR reaction mixture comprises a third set of primers targeting a different target polynucleotide sequence. It will be apparent to those skilled in the art that the third target polynucleotide sequence may be any target sequence known in the art.

In an additional, non-limiting embodiment, the primers in the third set of primers that hybridize to at least one end of the target polynucleotide sequence are a mixture of hot-start primers and non-hot-start primers. Typically, this mixture is also present in a ratio of hot-start primers to non-hot-start primers of between 1:9 and 19:1, inclusive. In other embodiments, any of the other ratios provided herein may be used, each of which constitutes a separate embodiment of the present invention. For example, the forward primers may be composed of a mixture of hot-start and non-hot-start primers.

In another embodiment, the reverse primers are composed of a mixture of hot-start and non-hot-start primers. In other embodiments, both the forward and reverse primers are composed of a mixture of hot-start and non-hot-start primers. Each possibility may be considered as being a separate embodiment of the present invention.

As provided herein in Examples 3 and 5, multiplex amplification reactions containing mixtures of hot-start and non-hot-start primers for targeting 3 different target polynucleotide sequences (amplicons) in multiplex qPCR, together with hot-start primers only for targeting a fourth amplicon, worked efficiently in multiplex PCR wherein certain amplification reactions are sensitive to mismatches and/or substances in the sample that may inhibit certain hybridizations.

In an additional, non-limiting embodiment, a PCR reaction mixture or method of the present invention contains three sets of primers, each capable of amplifying a different amplicon. In another embodiment, a PCR reaction mixture or method of the present invention contains four sets of primers, each capable of amplifying a different amplicon. In yet another, non-limiting embodiment, a PCR reaction mixture or method of the present invention contains five sets of primers, each capable of amplifying a different amplicon. In still another embodiment, a PCR reaction mixture or method of the present invention contains six sets of primers, each capable of amplifying a different amplicon. In yet another embodiment, a PCR reaction mixture or method of the present invention contains more than six sets of primers, each capable of amplifying a different amplicon. In yet another embodiment, a PCR reaction mixture or method of the present invention contains between 6-500, inclusive, sets of primers, each capable of amplifying a different amplicon. Examples 1-6 present PCR mixtures containing four sets of primers, each capable of amplifying a different DNA Target. As is known to those skilled in the art, instruments, such as the Rotor-Gene® Q (Qiagen) and LightCycler® (Roche Molecular Detection) enable performance of multiplex PCR reactions containing up to six sets of primers, and the FLEXMAP 3D™ (Luminex Corporation) enables multiplex PCR with up to 500 distinct PCR primers sets.

It has been mentioned that methods and compositions of the present invention comprise one or more mixtures of hot-start primers and non-hot-start primers against a particular polynucleotide sequence, which typically are present in a ratio of hot-start primers to non-hot-start primers of between 1:9 and 19:1, inclusive. In more specific embodiments, each mixture of hot-start primers and non-hot-start primers (in other words, the mixture directed against each end of a particular amplicon) is present in a ratio of hot-start primers to non-hot-start primers of between 1:5 and 19:1, inclusive. In other embodiments, each mixture of hot-start primers and non-hot-start primers is present in a ratio of hot-start primers to non-hot-start primers of between 1:4 and 14:1, inclusive. In other embodiments, each mixture of hot-start primers and non-hot-start primers is present in a ratio of hot-start primers to non-hot-start primers of between 1:3 and 14:1, inclusive. In other embodiments, each mixture of hot-start primers and non-hot-start primers is present in a ratio of hot-start primers to non-hot-start primers of between 1:2 and 14:1, inclusive. In other embodiments, each mixture of hot-start primers and non-hot-start primers is present in a ratio of hot-start primers to non-hot-start primers of between 1:1 and 14:1, inclusive. In other embodiments, each mixture of hot-start primers and non-hot-start primers is present in a ratio of hot-start primers to non-hot-start primers of between 1:1 and 9:1, inclusive. In other embodiments, each mixture of hot-start primers and non-hot-start primers is present in a ratio of hot-start primers to non-hot-start primers of between 2:1 and 14:1, inclusive. In other embodiments, each mixture of hot-start primers and non-hot-start primers is present in a ratio of hot-start primers to non-hot-start primers of between 2:1 and 9:1, inclusive. In other embodiments, each mixture of hot-start primers and non-hot-start primers is present in a ratio of hot-start primers to non-hot-start primers of between 3:1 and 9:1, inclusive. In other embodiments, each mixture of hot-start primers and non-hot-start primers is present in a ratio of hot-start primers to non-hot-start primers of between 4:1 and 9:1, inclusive. Each possibility may be considered as being a separate embodiment of the present invention.

In some embodiments, the divalent cation used in methods and compositions of the present invention is stored and/or provided separately from the other components of the reaction mixture, and may be withheld until after the template is added. In other embodiments, the divalent cation is provided together with the other components of the reaction mixture.

qPCR and Other Cycle Threshold Amplification Reaction Assays

In light of the disclosure provided herein, it will be appreciated by those skilled in the art that methods of the present invention are suitable for any type of PCR reaction, either quantitative (“real-time”) or non-quantitative. In certain embodiments, methods of the present invention are particularly suitable for threshold value amplification reactions, of which real-time PCR is provided as a non-limiting exemplary embodiment. “Threshold value amplification reaction” refers to a nucleic acid amplification reaction wherein the assay determines a threshold value such as for example the cycle number at which amplification of a particular target sequence above a threshold level is achieved. In one non-limiting embodiment, fluorescently labeled probes are utilized to monitor amplification. In another embodiment, any other means known in the art for monitoring amplification is utilized, such as a double-stranded DNA-binding dye, e.g., SYBR Green. Each possibility may be considered as being a separate embodiment of the present invention.

qpCR-Based Assays For Detecting Sequences of Interest in Test Samples

qPCR is well known in the art. qPCR reaction mixtures typically comprise the 4 naturally-occurring deoxynucleoside triphosphates (dNTPs); a divalent cation, and a polymerase enzyme. Most often, the divalent cation is a magnesium ion. In some embodiments, a thermophilic DNA polymerase enzyme is utilized. qPCR has been used for detecting human and animal target nucleic acid sequences and sequences specific to pathogens in a variety of applications.

One exemplary type of target polynucleotide sequences is a polynucleotide sequence characteristic of a pathogen of interest, typically assayed in a clinical specimen. Use of qPCR-based assays for detecting both pathogens in general and antibiotic-resistant pathogens in particular are well known in the art, and are described, for example, in U.S. Pat. No. 6,664,080 to Klaus Pfeffer, entitled “TaqMan™-PCR for the detection of pathogenic E. coli strains”; US Pat. App. No, 2009/0081663 to Yosef Paitan, entitled Methods, Compositions and Kits for Detection and Analysis of Antibiotic-Resistant Bacteria”; U.S. Pat. No. 6,329,138 to Hans De Beenhouwer et al, entitled “Method for detection of the antibiotic resistance spectrum of mycobacterium species”; U.S. Pat. No. 7,045,291 to Nancy Hanson et al, entitled “Multiplex PCR for the detection of AmpC beta-lactamase genes”; U.S. Pat. Nos. 5,994,066 and 6,001,564 to Bergeron et al, assigned to Creighton University; and International patent application WO/1996/008582 and US Pat. App. No. 2004/0185478, each to Bergeron et al. Each of these patents and applications is incorporated herein by reference.

Another specific application is the use of qPCR-based assays for detecting target polynucleotide sequences in forensic samples, for example DNA markers that can be used to determine the identity of a human. Such methods are well known in the art and are described inter alia in U.S. Pat. Nos. 6,673,541 and 5,861,504 and US patent application nos. 2009/0023603, 2006/0099620, and 2008/0286773, which are incorporated herein by reference. Often, several amplification reactions are run in parallel to make best use of very limited available DNA sample.

Another specific application is the use of qPCR-based assays for detecting target polynucleotide sequences by qPCR, for example nucleotide markers of disease and disease susceptibility such as BRCA1 and BRCA2. Such methods are well known in the art and described inter alia in U.S. Pat. Nos. 7,507,800 and 6,083,698 US patent application no. and 20050095592, which are incorporated herein by reference.

Hydration-Reduced PCR Reaction Mixtures

In light of the disclosure provided herein, those skilled in the art will appreciate that the compositions and methods of the present invention are compatible with both ordinary and storage-stabilized PCR reaction mixtures, such as but not limited to hydration-reduced PCR reaction mixtures. The reaction mixtures utilized in the Examples herein were treated to reduce hydration and were stored at room temperature until use. However, very similar, if not identical, results may be obtained with ordinary PCR reaction mixtures. Thus, in one embodiment, the PCR reaction mixtures of the present invention are hydration-reduced PCR reaction mixtures. In another embodiment, they are ordinary reaction mixtures. In another embodiment, they are any type of reaction mixtures known in the art. Each possibility may be considered as being a separate embodiment of the present invention.

Hydration-reduced PCR reaction mixtures that are ambient temperature-stabilized are further described in co-pending US patent application 2008/0050737. Such mixtures are prepared by hydration-reducing solutions containing DNA polymerase and/or dNTPs, and also containing a buffer compound containing at least one stabilizing agent, and are stored at a temperature between 25° C.-100° C., typically about 55° C. The stabilizing agent(s) may be inter cilia a sugar and a protein, for example sucrose and/or BSA. Typically, 1-20% sucrose and 0.5-3 mg/ml BSA are included. In another embodiment, any other type of hydration-reduced PCR mixture known in the art is utilized. In another embodiment, any other type of ambient temperature-stabilized PCR mixture known in the art is utilized. Each possibility may be considered as a separate embodiment of the present invention.

### Primers

The term “primer” as used herein refers to an oligonucleotide, either natural or synthetic, that is capable, upon forming a duplex with a polynucleotide template, of acting as a point of initiation of nucleic acid synthesis and being extended from one end along the template so that an extended double strand (duplex) is formed. In one embodiment, the polynucleotide template is a DNA molecule. In one embodiment, the polynucleotide template is an RNA molecule. Primers of the invention may or may not be detectably labeled. Primers may be hot-start or regular primers. Hot-start primers are described hereinbelow. The terms “regular primer” and “non-hot-start primer” refer to primers that are not hot-start primers as defined herein. Primers described as “for”, “directed to”, or “capable of amplifying” a particular target sequence are complementary to the ends of the target sequence, with the 3′ ends facing inward, such that the target sequence can be amplified in a PCR reaction.

The terms “nucleotide”, “nucleoside”, “nucleotide residue”, and “nucleoside residue” as used herein refer to deoxyribonucleotide or ribonucleotide residues, or other similar nucleoside analogues capable of serving as components of primers suitable for use in a PCR reaction. Such nucleoside and derivatives thereof are used as the building blocks of the primers described herein, except where indicated otherwise. Nothing in this application is mean to preclude the utilization of nucleoside derivatives or bases that have been chemical modified to enhance their stability or usefulness in a PCR reaction, provided that the chemical modification does not interfere with their recognition by DNA polymerase as deoxyguanine, deoxycytosine, deoxythymidine, or deoxyadenine, as appropriate.

In certain embodiments, nucleotide analogues that may be used in methods and compositions of the present invention include derivatives wherein the sugar is modified, as in 2′-O-methyl, 2′-deoxy-2′-fluoro, and 2′,3′-dideoxynucleoside derivatives, nucleic acid analogs based on other sugar backbones, such as threose, locked nucleic acid derivatives, bicyclo sugars, or hexose, glycerol and glycol sugars, nucleic acid analogs based on non-ionic backbones, such as “peptide nucleic acids”, these nucleic acids and their analogs in non-linear topologies, such as dendrimers, comb-structures, and nanostructures, and these nucleic acids and their analogs carrying tags (e.g., fluorescent, functionalized, or binding) bound to their ends, sugars, or nucleobases.

One non-limiting example of non-classical nucleotide analogues suitable for use in methods and compositions of the present invention are locked nucleic acid (LNA) nucleotide analogues. Certain embodiments of LNA nucleotide analogues are bicyclic nucleic acid analogs that contain one or more 2′-O, 4′-C methylene linkages, which effectively lock the furanose ring in a C3′-endo conformation. This methylene linkage “bridge” restricts the flexibility of the ribofuranose ring and locks the structure into a rigid bicyclic formation. Because of its unique structural conformation, oligonucleotides comprising LNA nucleotide analogues demonstrate a much greater affinity and specificity to their complementary nucleic acids than do natural DNA counterparts. LNAs typically hybridize to complementary nucleic acids even under adverse conditions, such as under low salt concentrations. LNA nucleotide analogues are commercially available, and are described, inter alia, in U.S. Pat. Nos. 6,130,038, 6,268,490, and 6,670,461.

Another non-limiting example of non-classical nucleotide analogues suitable for use in methods and compositions of the present invention are peptide nucleic acid (PNA) nucleotide analogues. In certain embodiments of PNA nucleotide analogues, the negatively charged sugar-phosphate backbone of DNA is replaced by a neutral polyamide backbone composed of N-(2-aminoethyl) glycine units (see illustration below, wherein B represents a nucleoside base). The chemical configuration of PNA typically enables the nucleotide bases to be positioned in approximately the same place as in natural DNA, allowing PNA to hybridize with complementary DNA or RNA sequence. PNA nucleotide analogues are commercially available, and are described, inter alma, in the PCT Applications having the Publication Nos. WO 92/20702, WO 92/20703 and WO 93/12129.

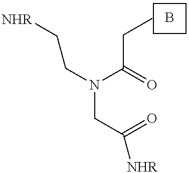

Another non-limiting example of non-classical nucleotide analogues suitable for use in methods and compositions of the present invention are glycol nucleic acid (GNA) nucleotide analogues (Zhang, L et al (2005) A simple glycol nucleic acid. J. Am, Chem. Soc. 127:4174-4175). Certain embodiments of GNA nucleotide analogues have an acyclic propylene glycol phosphodiester backbone and have one of the structures below, wherein B represents a nucleoside base:

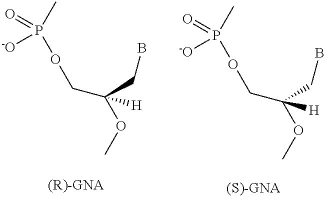

Another non-limiting example of non-classical nucleotide analogues suitable for use in methods and compositions of the present invention are threose nucleic acid (TNA) nucleotide analogues (Wu et al, Organic Letters, 2002, 4(8):1279-1282). Certain embodiments of TNA nucleotide analogues have the structure below, wherein B represents a nucleoside base:

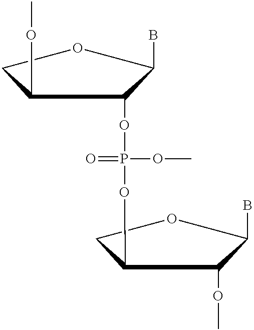

Another non-limiting example of non-classical nucleotide analogues suitable for use in methods and compositions of the present invention are bicyclic and tricyclic nucleoside analogs (Steffens et al, Helv Chim Acta (1997) 80:2426-2439; Steffens et al, J Am Chem Soc (1999) 121: 3249-3255; Renneberg et al, J Am Chem Soc (2002) 124: 5993-6002; and Renneberg et al, Nucl Acids Res (2002) 30: 2751-2757).

Another non-limiting example of non-classical nucleotide analogues suitable for use in methods and compositions of the present invention are phosphonomonoester nucleic acids which incorporate a phosphorus group in the backbone, for example analogues with phosphonoacetate and thiophosphonoacetate internucleoside linkages (US Pat. App. No. 2005/0106598; Sheehan et al, Nucleic Acids Res (2003); 31(14):4109-18). In other embodiments, a cyclobutyl ring replaces the naturally occurring furanosyl ring.

In other embodiments of non-classical nucleotide analogues suitable for use in methods and compositions of the present invention, the base is modified. A representative, non-limiting list of modified nueleobases includes 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl

(—C═C—CH₃)

uracil and cytosine and other alkynyl derivatives of pyrimidine bases, 6-azo uracil, cytosine and thymine, 5-uracil(pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine (1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine
(e.g. 9-(2-aminoethoxy)-H-pyrimido(5,4-b)(1,4)benzoxazin-2(3H)-one), carbazole cytidine (2H-pyrimido(4,5-b)indol-2-one), and pyridoindole cytidine
(H-pyrido(3′,′: 4,5)pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808; those disclosed in The Concise Encyclopedia Of Polymer Science And Engineering, pages 858-859, Kroschwitz, J. I., ed. John Wiley & Sons, 1990; those disclosed by Englisch et al., Angewandte Chemie, International Edition, 1991, 30, 613; and those disclosed by Sanghvi, Y. S., Chapter 15, Antisense Research and Applications, pages 289-302, Crooke, S. T. and Lebleu, B., ed., CRC Press, 1993. Certain of these nucleobases are known to those skilled in the art as suitable for increasing the binding affinity of the compounds of the invention. These include 5-substituted pyrimidines, 6-azapyrimidines and N-2, N-6 and O-6 substituted purines, including 2-aminopropyladenine, 5-propynyluracil and 5-propynylcytosine. Modified nucleobases and their use are described, inter cilia, in U.S. Pat. No. 3,687,808, U.S. Pat. No. 4,845,205; U.S. Pat. No. 5,130,302; U.S. Pat. No. 5,134,066; U.S. Pat. No. 5,175,273; U.S. Pat. No. 5,367,066; U.S. Pat. No. 5,432,272; U.S. Pat. No. 5,457,187; U.S. Pat. No. 5,459,255; U.S. Pat. No. 5,484,908; U.S. Pat. No. 5,502,177; U.S. Pat. No. 5,525,711; U.S. Pat. No. 5,552,540; U.S. Pat. No. 5,587,469; U.S. Pat. No. 5,594,121, U.S. Pat. No. 5,596,091; U.S. Pat. No. 5,614,617; U.S. Pat. No. 5,645,985; U.S. Pat. No. 5,830,653; U.S. Pat. No. 5,763,588; U.S. Pat. No. 6,005,096; U.S. Pat. No. 5,681,941; and U.S. Pat. No. 5,750,692.

Another non-limiting example of non-classical nucleotide analogues suitable for use in methods and compositions of the present invention are polycyclic heterocyclic compounds in place of one or more of the naturally-occurring heterocyclic base moieties. A number of tricyclic heterocyclic compounds have been previously reported. These compounds are routinely used in antisense applications to increase the binding properties of the modified strand to a target strand. The most studied modifications are targeted to guanosines hence they have been termed G-clamps or cytidine analogs. Representative cytosine analogs that make 3 hydrogen bonds with a guanosine in a second strand include 1,3-diazaphenoxazine-2-one (Kurchavov, et al., Nucleosides and Nucleotides, 1997, 16, 1837-1846), 1,3-diazaphenothiazine-2-one, (Lin, K.-Y.; Jones, R. J.; Matteucci, M. J. Am. Chem. Soc. 1995, 117, 3873-3874) and 6,7,8,9-tetrafluoro-1,3-diazaphenoxazine-2-one (Wang, J.; Lin, K.-Y., Matteucci, M. Tetrahedron Lett. 1998, 39, 8385-8388). Incorporated into oligonucleotides these base modifications were shown to hybridize with complementary guanine and the latter was also shown to hybridize with adenine and to enhance helical thermal stability by extended stacking interactions (also see U.S. Pre-Grant Publications 2003/0207804 and 2003/0175906).

In another embodiment, a primer utilized in methods and compositions of the present invention may comprise one or more universal nucleosides. Non-limiting examples of universal nucleosides are 5-nitroindole and inosine, as described inter alia in US 2009/0325169 and 2010/0167353.

### Hot-Start Primers

The term “hot-start primers” as used herein refers to primers that are inactive, or have reduced activity, until exposure to elevated temperatures. Numerous types of hot-start primers exist in the art and are well known to the skilled artisan, including, but not limited to, primers comprising a modified nucleotide at the 3′ end of the primer (see, e.g., U.S. Pat. No. 6,482,590), primers possessing a stem-loop or hairpin structure (see, e.g., U.S. Pat. App. No, 2007/0128621), primers containing a heat-removable chemical modification at the 3′ end of the primer (see, e.g., U.S. Pat. App. No. 2007/0281308), primers bound to blocking oligonucleotides (see, e.g., U.S. Pat. No. 5,712,386), and ribo-primers (see, e.g., U.S. Pat. App. Nos. 2009/0325169 and 2010/0167353). A subset of hot-start primers are primers that gradually generate extendible primers over the course of a multi-cycle amplification. These types of primers confer increased reaction specificity at every cycle, as opposed to hot-start polymerase and certain types of hot-start primers that generate a large amount of extendible primer during the denaturation step.

In certain other embodiments, hot-start primers useful in the present invention are those that contain a modification so as to generate an extendible primer only when hybridized to complementary sequence while at elevated temperatures. One exemplary, non-limiting sub-class of this type of hot-start primers, referred to herein collectively as “ribo-primers,” are those that are reversibly chemically modified such that they are unable to serve as primers for DNA polymerase. Ribo-primers are also substrates, when hybridized to a DNA target sequence, for cleavage by an endonuclease, for example a RNaseH2 endonuclease, which results in removal of the chemical modification, as exemplified herein. In some embodiments, such modified primers may comprise an RNA moiety, which serves as the RNase H cleavage site. Various embodiments of ribo-primers are further described hereinbelow.

The blocking modification (alternatively referred to herein as an “inactivating chemical modification”) of a ribo-primer is preferably located 3′ (downstream) to the cleavage site of an RNaseH2 endonuclease. More preferably, the blocking modification is located at the 3′-end of the ribo-primer. In some embodiments, the blocking modification may be positioned up to about 10 residues from the 3′ end of the oligonucleotide of the invention, followed by nucleotide sequence that is not either mostly non-complementary or entirely non-complementary to the target. In any case, a “ribo-primer” as defined herein contains a modification that precludes its suitability as a primer for elongation and that is reversed by the action of an endonuclease (also referred to herein as an “activating enzyme”). Typically the reversal involves physical removal of the modification.

In other embodiments, the term “ribo-primers” refers to variants of standard DNA oligonucleotide primers, containing the following modifications: At least one RNA residue is added at the 3′-end of a standard DNA oligonucleotide primer, followed by at least three additional DNA residues and a blocking group. The primer sequence formed by the RNA and DNA residues of the ribo-primers exhibits partial or complete complementarity with the sequence of the target DNA. A single RNA residue within a ribo-primer bound to a DNA target is typically sufficient to render the heteroduplex a substrate for endonuclease cleavage by RNaseH2, with the cleavage occurring immediately 5′ to the RNA residue, removing the RNA residue itself and all residues and modifications 3′ to the RNA residue. Individuals skilled in the art will recognize that the presence within the ribo-primer of one or more additional complementary RNA residues positioned 3′ to the first RNA residue will typically not affect the ability of the primer to serve as a substrate for endonuclease cleavage by RNaseH2.

In another embodiment, a ribo-primer used in a method or composition of the present invention contains a single 2′-fluoro-modified residue in an internal position thereof. Cleavage by RNase H2 typically occurs on the 5′ side of the 2′-fluoro-modified residue. In another embodiment, the ribo-primer comprises an RNase H2 cleavage domain comprising two adjacent 2′-fluoro-modified residues. In this embodiment, cleavage occurs primarily between the 2′-fluoro-modified residues. In one embodiment, the one or more 2′-fluoro-modified residues are DNA residues. In another embodiment, the one or more 2′-fluoro-modified residues are RNA residues. Each possibility represents a separate embodiment of the present invention.

Other types of modified residues can also be present in a ribo-primer, including but not limited to 2′-O-alkyl-modified residues, preferably 2′-O-methyl-modified residues, locked nucleic acids (LNA), 2′-ENA residues (ethylene nucleic acids), 2′-alkyl-modified residues, 2′-amino-modified residues, and 2′-thio-modified residues. In some embodiments, these modifications may be made to residues other than the RNA residue at the intended site of enzymatic cleavage, either immediately adjacent to the RNA residue or at positions further removed therefrom. In other embodiments, these modified residues may be introduced in place of the RNA residue at the intended site of enzymatic cleavage. In one more specific embodiment, a 2′-fluoro-modified RNA residue is used in combination with a 2′ LNA-modified RNA residue.

In more specific embodiments, the 2′-hydroxy group of an RNA residue is replaced with one of the above-described alternative functional groups. In other embodiments, the phosphate group immediately 3′ to the RNA residue in the intended RNase H is replaced with a nuclease-resistant linkage to prevent aberrant cleavage by an RNase H enzyme. In other embodiments, the oligonucleotide is modified with a nuclease-resistant linkage or residue further downstream from the 3′-phosphate group of the RNA residue or on the 5′-side of the RNA residue. In some embodiments, the nuclease-resistant linkage that is utilized is selected from a phosphorothioate, phosphorodithioate, methylphosphonate, or boronate linkage. In another embodiment, an abasic residue such as a C3 spacer is inserted immediately 3′ to the RNA residue to prevent aberrant cleavage. Alternatively one or both of the hydrogen atoms on the 5′ carbon of the adjacent residue can be replaced with bulkier substituents such as methyl groups to inhibit background cleavage of a ribonucleotide residue. Combinations of these various modifications may also be employed. The above modifications are well-known in the art. Typically, such modifications are most useful for PCR reactions used for mismatch discrimination, such as AS-PCR (described hereinbelow).

In certain embodiments, alternative divalent cations such as Mn²⁺, Ni²⁺ or Co²⁺, with or without Mg²⁺, are incorporated into an assay buffer indicated for use with a ribo-primer. In certain embodiments of the invention, when such alternative divalent cations are present, enhanced cleavage by RNase H2 is achieved. In one more specific embodiment, Mn²⁺ in combination with Mg²⁺ is included in a buffer intended for use with a ribo-primer containing two adjacent 2′-fluoro-modified residues.

Various embodiments of ribo-primers, including the embodiments mentioned herein, are described in US App. Pub. Nos. 20090325169 and 20100167353.

The position of the ribonucleotide or other modified residue affects the ability of a ribo-primer to serve as a substrate for RNase H2. Preferably, the ribonucleotide or other modified residue of a ribo-primer used in methods and compositions of the present invention is flanked on its 3′-side by a total of about 1-10 DNA residues. In other embodiments, a total of about 3-6 DNA residues flank the ribonucleotide or other modified residue on its 3′-side. In other embodiments, a total of about 2-5 DNA residues flank the ribonucleotide or other modified residue on its 3′-side. In other embodiments, a total of about 3-5 DNA residues flank the ribonucleotide or other modified residue on its 3′-side. In other embodiments, a total of about 4-5 DNA residues flank the ribonucleotide or other modified residue on its 3′-side. In other embodiments, about 4-5 DNA residues that are complementary to the target sequence immediately flank the ribonucleotide or other modified residue on its 3′-side. Optionally, one or more additional DNA residues not complementary to the target sequence may be present 3′ to these 4-5 complementary DNA residues.

In other embodiments, at least 8 deoxyribonucleotides that are complementary to the target sequence flank the ribonucleotide or other modified residue on its 5′-side. In another embodiment, at least 10 DNA residues that are complementary to the target sequence flank the ribonucleotide or other modified residue on its 5′-side. In other embodiments, at least 12 DNA residues that are complementary to the target sequence flank the ribonucleotide or other modified residue on its 5′-side. In other embodiments, 8-50 DNA residues that are complementary to the target sequence flank the modified residue on its 5′-side. In another embodiment, 10-50 DNA residues that are complementary to the target sequence flank the modified residue on its 5′-side. In another embodiment, 12-50 DNA residues that are complementary to the target sequence flank the modified residue on its 5′-side. Optionally, one or more additional DNA residues not complementary to the target sequence may be present 5′ to these complementary DNA residues that are located 5′ to the modified residue.

As mentioned hereinabove, in certain embodiments, hot-start primers of the present invention contain a reversible chemical modification, for example a 3′ blocking group, that precludes their suitability to serve as a primer for DNA polymerase, until cleavage by a nuclease enzyme such as RNase H. In certain embodiments, the nuclease requires correct base pairing in the vicinity of the cleavage site at elevated temperature, in order to become activated. The term “elevated temperatures” in this context refers, in various embodiments, to a temperature over 50° C., over 60° C., 70° C., or over 80° C. In other preferred embodiments, the nuclease exhibits activity at temperatures between 50-80° C. and does not exhibit appreciable activity at 25° C. In other preferred embodiments, the nuclease exhibits activity of the annealing temperature of the reaction and does not exhibit appreciable activity at 25° C. In certain other preferred embodiments, the nuclease is thermostable and retains activity following a 30-minute incubation at a temperature of 95° C. Each possibility may be considered as being a separate embodiment of the present invention.

### Blocking Modifications

The C3 Spacer exemplified herein prevents primer extension prior to cleavage, thereby reducing non-specific primer extension. Individuals skilled in the art will recognize in light of the disclosure provided herein that the C3 Spacer is but an exemplary example of a blocking moiety, and that various alternative spacer modifications and other common oligonucleotide modifications readily available from commercial suppliers of custom synthesized oligonucleotides, such as Integrated DNA Technologies, Inc. (Coralville Iowa) and Eurogentec North America Inc. (San Diego Calif.), are capable of blocking 3′ extension of a primer by DNA polymerase and can be used in place or in addition to the C3 Spacer. Such alternative blocking modifications include, but are not limited to, Hexanediol, Spacer 9, Spacer 18, 2′,3′-Dideoxy-C, Dideoxy-A Dideoxy-G, Dideoxy-T, a 3′ deoxyribonucleotide residue (for example deoxyribo-C, A, G, or T, or cordycepin), non-nucleotide linkages, alkane-diol modifications (inter cilia as described in U.S. Pat. No. 5,554,516), 3′ hydroxyl substitutions (for example 3′-phosphate, 3′-triphosphate or 3′-phosphate diesters with alcohols such as 3-hydroxypropyl), addition of a 2′3′-cyclic phosphate, 2′ hydroxyl substitutions of a terminal RNA residue (for example phosphate or sterically bulky groups such as triisopropyl silyl (TIPS) or tert-butyl dimethyl silyl (TBDMS)), and 2′-alkyl silyl groups such as TIPS and TBDMS substituted at the 3′-end of an oligonucleotide, for example as described in U.S. patent application 2007/0218490. Bulky substituents can also be incorporated on the base of the 3′-terminal residue of the oligonucleotide to block primer extension. In other embodiments, the blocking modification may also serve as a fluorescence quencher such as IBFQ (Iowa Black FQ, Integrated DNA Technologies, Coralville, Iowa), or any other suitable quencher moiety. Those of skill in the art will understand in light of the disclosure provided herein that a ribo-primer used in the present invention may further include one or more additional DNA nucleotide residues 3′ to the RNA residue or 3′ to the blocking group, based on known PCR primer design considerations, such as a desire to increase or decrease primer specificity, binding affinity or melting temperature.

The C3 Moiety present in the ribo-primers utilized herein has the formula C₃₃H₄₃N₂O₅P and has the following structure:

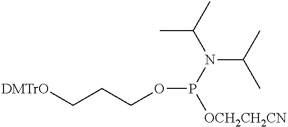

In other embodiments, the blocking group of a ribo-prime is a single C3 Moiety (described hereinbelow). In certain embodiments, the single RNA residue is followed by a C3 Moiety, which is followed by a second C3 Moiety which is followed by one or more additional DNA residues.

### RNase H Enzymes

Some embodiments of ribo-primers utilized in methods and compositions of the present invention are activated using an RNase H2 enzyme, which is typically a thermostable and thermophilic RNase H2 enzyme. Thermostable RNase H2 enzymes and methods for using same are well known in the art (Haruki et al, Gene Cloning and Characterization of Recombinant RNase HII from a Hyperthermophilic Archaeon. Journal of Bacteriology, December 1998, p. 6207-6214.) An exemplary, non-limiting thermostable RNase H2 enzyme is P. abyssi Ribonuclease H2 enzyme, utilized in the Examples herein. P. abyssi RNaseH2 is a thermo-stable and thermophilic RNaseH enzyme. The RNaseH enzyme binds to regions where a ribonucleotide is bound to a deoxyribonucleotide. Once bound, the enzyme cleaves immediately 3′ of the RNA residue. In various embodiments, the hot start/thermophilic properties of an RNase H2 enzyme used in conjunction with ribo-primers in methods and compositions of the present invention may be either intrinsic to the enzyme or a result of reversible chemical inactivation or a blocking antibody, as is well known in the art.

In some embodiments, a detergent is included in the reaction buffer to increase the efficiency of RNase H2. In some more specific embodiments, the detergent is selected from Triton-X100, Tween-20, and cetyltrimethylammonium bromide (CTAB).

In PCR and qPCR reaction incubations, RNaseH2 typically becomes activated once a high temperature is reached, enabling it to cleave its substrate, thereby liberating the residues 3′ to the RNA residue and the C3 Spacer and enabling amplification. Since RNaseH2 requires that the primer/target match be sufficient to form a strong DNA-RNA duplex in the region near the ribonucleotide, the ribo-primer reaction is usually more specific than a standard oligonucleotide primer. Additionally, since the enzyme is only activated at high temperatures, the ribo-primer reaction typically reduces non-specific primer extension, thus reducing undesired nucleotide extension and amplification.

### Probes

The term “probe” as used herein refers to an oligonucleotide, either natural or synthetic, that is generally detectably labeled and used to identify complementary nucleic acid sequences by hybridization. Primers and probes of use in the invention may have identical or different sequences. “Probe suitable for real-time PCR” refers to any probe that emits a detectable signal in real-time in the presence of the target sequence, including those described in U.S. Pat. Nos. 5,925,517, 6,037,130, 6,103,476, 6,150,097, 6,461,817 and 7,385,043, which are incorporated herein by reference.

In another embodiment, the probe is a dual-modified oligonucleotide, as utilized in the Examples herein. Dual-modified oligonucleotides are well known in the art, and are described, inter alia, in International patent application WO 2008/063194 and in US App. Pub. Nos. 2009/0068643, 2009/0325169, and 2010/0167353. These include, but are not limited to, TaqMan® Probes, Eclipse™ and Molecular Beacons. An exemplary, non-limiting type of suitable probe is a Molecular Beacon. Use of Molecular Beacons is well known in the art, and is described, inter alia, in Tyagi S and Kramer F R (1996) Molecular beacons: probes that fluoresce upon hybridization. Nat Biotechnol 14, 303-308. Molecular Beacons and other probes suitable for real-time PCR typically include a fluorescent reporter molecule at the 5′-end and a quencher molecule at the 3′-end. Probes modified with any one of an extensive group of fluorophores are commercially available and referenced in the Product Catalogs of suppliers such as Integrated DNA Technologies, Inc. (Coralville Iowa), Eurogentec North America Inc. (San Diego Calif.) and Biosearch Technologies Inc. (Novato, Calif.). Non-limiting examples include FAM, HEX, TET, ROX, Texas Red, Cy 5, TYE 665, TYE 563, Quasar carboxylic acids and Quasar active esters. As is well known to those skilled in the art, the selection of an appropriate quencher moiety is determined by the fluorescence emission of the probe's fluorophore and, includes, but is not limited to Black Hole Quencher-1, Black Hole Quencher-2, Black Hole Quencher-3, Iowa Black FQ, Iowa Black RQ-Sp, Dabcyl, Deep Dark Quencher I, Deep Dark Quencher II and Deep Dark Quencher III.

In other embodiments, the probes are any other type of probes known in the art. Those of skill in the art will understand in light of the disclosure provided herein that a variety of types of probes may be utilized in amplification reactions of the present invention without appreciably affecting the performance of the invention, and that any combination of different fluorophores and quenchers can be readily used for each of the probes in the reaction mixture. Each possibility may be considered as being a separate embodiment of the present invention.

### Target Sequences

The terms “target nucleic acid”, “target polynucleotide”, “target polynucleotide sequence”, “target polynucleotide molecule”, and “target gene” are used interchangeably and synonymously herein and refer to the nucleotide sequence on the template nucleic acid strand to which the primer is intended to hybridize. In various embodiments, the target sequence may comprise an RNA or DNA strand. The terms may refer to a portion of a target gene or to a target gene in its entirety. In another embodiment, a method or kit of the present invention utilizes primers for amplifying a target gene or polynucleotide sequence characteristic of (specific for) a species of interest. Those skilled in the art will readily understand that a pair of primers is capable of amplifying a particular target polynucleotide sequence in a PCR reaction if they hybridize to opposite ends of the sequence in an inwardly-pointing direction. In certain embodiments, the gene or polynucleotide sequence may be any gene or polynucleotide sequence whose sequence in the pathogen of interest is unique among common microorganisms. The term “species-specific gene” is used generically herein to refer to any species-specific sequence, whether a gene or intergenic region. In some embodiments, the sequences that hybridize with the primers are preferably unique in the reaction mixture.

### Methods for Detecting Target Sequences of Interest in a Test Sample

In another embodiment, the present invention provides a method of detecting two or more target polynucleotide sequences in a test sample, wherein said test sample has been derived from a clinical specimen and has not been extensively purified, the method comprising the steps of: (a) amplifying one or more nucleic acids from the test sample using a PCR reaction mixture; and (b) detecting the amplified PCR product, wherein the PCR reaction mixture comprises: i) a first set of primers, comprising at least one forward primer and at least one reverse primer, that is capable of amplifying a first sequence of said first target polynucleotide sequences; and ii) a second set of primers, comprising at least one forward primer and at least one reverse primer, that is capable of amplifying a first sequence of said second target polynucleotide sequences, wherein at least one of said forward primer and said reverse primer of said first set of primers is provided as a mixture of hot-start primers and non-hot-start primers. Preferably, the sets of primers used to amplify both of the target polynucleotide sequences comprise a forward and/or or said reverse primer that is provided as a mixture of hot-start primers and non-hot-start primers. Typically, the reaction mixture further comprises one or more of a nucleic acid polymerase enzyme, dNTPs, and a divalent cation, most often a magnesium ion. The hot-start primers may contain an inactivating chemical modification that is reversed by the action of the activating enzyme present in the reaction mixture, or may be other types of hot-start primers known in the art. In certain embodiments, the PCR reaction mixture further comprises a probe or double-stranded DNA binding dye suitable for real-time PCR. It will be understood by those skilled in the art that the PCR reaction mixture used in this method may further comprise additional sets of primers for amplification of addition target sequences.

In another embodiment, the present invention provides a method of detecting a species-specific target polynucleotide sequence in a test sample, the method comprising the steps of: (a) performing PCR on nucleic acids from the test sample using a reaction mixture of the present invention, wherein the PCR comprises at least one amplification reaction that targets a sequence whose sequence in the pathogen of interest is unique among common microorganisms; and (b) detecting the amplified PCR product.

In another embodiment, the present invention provides a method of detecting a species-specific target polynucleotide sequence in a test sample, the method comprising the steps of: (a) of performing real-time PCR on nucleic acids from the test sample using a reaction mixture of the present invention, wherein one amplification reaction in the PCR targets a sequence whose sequence in the pathogen of interest is unique among common microorganisms; (b) measuring the signal generated by the probe that detects the species-specific gene crosses a preset intensity; and (c) comparing the cycle number to a reference standard. In certain embodiments of this method, the reference standard relates to the signal cycle number at which the signal generated by the probe that detects the species-specific gene crosses a preset intensity (the Cycle Threshold” or “Ct value”). In more specific embodiments, if the Ct value is equal to or below the reference standard, then the sequence is deemed to be present in the test sample. In another embodiment, the present invention provides a kit for detecting a target sequence in a test sample. Each possibility may be considered as being a separate embodiment of the present invention.

In various embodiments, the target sequence of a kit of the present invention may be a pathogen-specific gene, human DNA marker or animal DNA marker, Detection of pathogen-specific sequences by PCR is well known in the art and is described, inter alia, in references cited hereinbelow.

“Nucleic acids from the test sample” refers to nucleic acids derived from the test sample of interest. It will be understood by those skilled in the art that test samples containing intact cells will be typically subject to a lysis procedure prior to performing the PCR reaction. In certain embodiments, the sample lysate has not been subjected to a nucleic acid purification procedure prior to the amplification reaction. In other embodiments, the sample lysate may have been subjected to a crude nucleic acid purification procedure, but not an extensive one. As provided herein, methods of the present invention overcome difficulties encountered with amplification of non-purified nucleic acid samples.

In some embodiments, a kit of the present invention is indicated for nucleic acid samples that have not necessarily been subjected to extensive purification.

As used herein, reference to nucleic acids that are “not extensively purified” or have not been “subjected to extensive purification” is intended to include nucleic acids that are extracted from lysed cells but that are not separated from the majority of the non-nucleic acid, soluble cellular components. This includes nucleic acids that are obtained by pelleting cells from a clinical or other type of sample, such as a bacterial culture; lysing those cells; and removing the insoluble cellular components from the lysed cells, such as cell wall components, for instance by centrifugation or filtration.

In certain embodiments, nucleic acids that are “not extensively purified” will have an A₇₆₀/A₂₈₀ ratio of less than 2.0 (the A₂₆₀/A₂₈₀ ratio is a measure of the purity of a nucleic acid preparation). In another embodiment, methods and compositions of the present invention are suitable for use with samples having an A₂₆₀/A₂₈₀ ratio of less than 1.9. In another embodiment, a kit of the present invention is suitable for use with samples having an A₂₆₀/A₂₈₀) ratio of less than 1.8. In another embodiment, a kit of the present invention is suitable for use with samples having an A₂₆₀/A₂₈₀ ratio of less than 1.7. In another embodiment, a kit of the present invention is suitable for use with samples having an A₂₆₀/A₂₈₀ ratio that is between 1.0-2.0. In another embodiment, a kit of the present invention is suitable for use with samples having an A₂₆₀/A₂₈₀ ratio that is between 1.0-1.9. In another embodiment, a kit of the present invention is suitable for use with samples having an A₂₆₀/A₂₈₀ ratio that is between 1.0-1.8. In another embodiment, a kit of the present invention is suitable for use with samples having an A₂₆₀/A₂₈₀ ratio that is between 1.0-1.7. Each possibility may be considered as being a separate embodiment of the present invention.

### Test Samples

The term “test sample” as used herein refers to any sample suspected of containing a target sequence, for instance a sample suspected of containing a pathogen of interest or human or animal DNA marker of interest. In certain embodiments, the test sample is a clinical specimen from a mammal. In certain other embodiments, the test sample is a clinical specimen from a human. The term “clinical specimen” as used herein refers alternatively to a specimen obtained from processing a body fluid, tissue, or any type of biopsy from a mammal. As provided herein, clinical specimens from mammals in general and humans in particular may contain a substance that inhibits hot-start primer PCR, such as polysaccharide or mucus (for example, nasal, sputum, throat, or vaginal swabs), especially when there is a weakened primer-template bonding, such as the presence of primer-template mismatch in the vicinity of the RNA residue of a hot-start primer. In a non-limiting exemplary example, the primer-template mismatch is the DNA residue immediately 5′ to the RNA residue within a hot-start primer.

In certain embodiments, the clinical specimen is a body fluid. In another embodiment, the clinical specimen is nasal fluid. In another embodiment, the clinical specimen is a nasal swab. In another embodiment, the clinical specimen is a swab from an armpit. In another embodiment, the clinical specimen is a swab from a groin. In another embodiment, the clinical specimen is whole blood. In another embodiment, the clinical specimen is serum. In another embodiment, the clinical specimen is plasma. In another embodiment, the clinical specimen is cerebrospinal fluid. In another embodiment, the clinical specimen is urine. In another embodiment, the clinical specimen is lymph fluid. In another embodiment, the clinical specimen is tears. In another embodiment, the clinical specimen is saliva. In another embodiment, the clinical specimen is milk of a subject. In another embodiment, the clinical specimen is amniotic fluid. In another embodiment, the clinical specimen is an external secretion of the respiratory tract. In another embodiment, the clinical specimen is an external secretion of the intestinal tract. In another embodiment, the clinical specimen is an external secretion of the genitourinary tract. In another embodiment, the clinical specimen is selected from the group consisting of nasal fluid, vaginal secretions, whole blood, serum, plasma, cerebrospinal fluid, urine, lymph fluids, tears, saliva, milk, amniotic fluid and an external secretion of the respiratory, intestinal or genitourinary tract of a subject in need of testing for a target sequence of interest. Each possibility may be considered as being a separate embodiment of the present invention.

In another embodiment, the clinical specimen is a tissue from a biopsy of a subject. Typically, the tissue will have been treated appropriately (for example, by homogenization), to render it a substrate for PCR amplification. In certain embodiments, the tissue is white blood cells. In certain embodiments, the tissue is a malignant tissue. In certain embodiments, the tissue is chorionic villi. In certain embodiments, the tissue is selected from the group consisting of white blood cells, malignant tissues, and chorionic villi. In certain embodiments, the tissue comprises a cell type selected from the group consisting of white blood cells, malignant tissues, and chorionic villi. In certain embodiments, the tissue consists essentially of a cell type selected from the group consisting of white blood cells, malignant tissues, and chorionic villi. Each possibility may be considered as being a separate embodiment of the present invention.

In another embodiment, the test sample is used for forensic purposes and contains bloodstains on FTA cards that contain inhibitors impregnated on the card, tissue, skin, hair follicles, saliva and cigarette butts.

### Methods for Detecting Pathogens in a Clinical Specimen

One specific embodiment of the present invention is a method of detecting an antibiotic-resistant strain of a pathogen in a clinical specimen, the method comprising the steps of: (a) performing real-time PCR on nucleic acids from the test sample, wherein the PCR reaction mixture is a reaction mixture of the present invention; (b) determining the Ct values of the signals generated by the probes that detect a pathogen-specific sequence, whether a gene or intergenic region (herein referred to as a “pathogen-specific gene”), and a polynucleotide sequence that confers antibiotic resistance (herein referred to as an “antibiotic resistance gene”); and (c) comparing the Ct value of the pathogenic-specific gene to the Ct value of the antibiotic resistance gene. In another embodiment, the method further comprises the steps of amplifying a “bridging region” (a region connecting the usual point of insertion of an element containing an antibiotic resistance gene [the “insertion point”] and a known location in the genome of the target pathogen) and determining the Ct value of the bridging region. In another embodiment, the present invention provides a kit for detecting an antibiotic-resistant strain in a test sample. Each possibility may be considered as being a separate embodiment of the present invention.

Non-limiting embodiments of pathogen-specific genes that may be used in the above method are nuc, Sa442, and femB of S. aureus. Pathogen-specific polynucleotide sequences and primers for amplifying same are well-known in the art, and are described, inter alia, in US Pat. App. No. 2009/0081663. A non-limiting example of an antibiotic-resistance gene is mecA (GenBank Accession No. AB097677). Those of skill in the art will understand that genes associated with antibiotic resistance may be located on a cassette or plasmid or may be integrated into a chromosome of the pathogen. A non-limiting example of a bridging region that may be utilized is SCCmec:orfX. Bridging regions are well known in the art, and are described, inter alia, in Cuny and Witte (PCR for the identification of methicillin-resistant Staphylococcus aureus (MRSA) strains using a single primer pair specific for SCCmec elements and the neighbouring chromosome-borne orfX. Clin Microbiol Infect. 2005; 11(10):834-7). It will be understood by those skilled in the art that any pathogen-specific gene, antibiotic-resistance gene, or bridging region known in the art may be used in the above method. Each possibility may be considered as being a separate embodiment of the present invention.

In the above method, the target pathogen (“pathogen of interest”) may be, in certain embodiments, a bacterium. In other embodiments, the target pathogen is a virus. In other embodiments, the target pathogen is a fungus. In other embodiments, the target pathogen is a protozoon. In other embodiments, the target pathogen is an antibiotic-resistant bacterium. In other embodiments, the target pathogen is an antibiotic-resistant virus. In other embodiments, the target pathogen is an antibiotic-resistant fungus. In other embodiments, the target pathogen is an antibiotic-resistant protozoon. In certain embodiments, as exemplified herein, the target pathogen is S. aureus. In certain other embodiments, as exemplified herein, the target pathogen is methicillin-resistant S. aureus (MRSA). In certain other embodiments, the target pathogen is VRSA (Vancomycin-resistant S. aureus), VRE (Vancomycin-resistant Enterococcus), Hepatitis C Virus (HCV), or Clostridium Difficile. In other embodiments, the target pathogen is any target pathogen known in the art. Each possibility may be considered as being a separate embodiment of the present invention.

Antibiotic resistance genes that may be targeted by the above methods include, by way of non-limiting example, mecA, vanA, vanB, vanC1, vanC2 vanC3, vanD, vanE, and vane. Each possibility may be considered as a separate embodiment of the present invention.

In another embodiment, the antibiotic resistance gene is a gene that confers resistance to one or more antibiotic agents selected from the group consisting of methicillin, vancomycin, linezolid, a penicillin-class antibiotic, a cephalosporin-class antibiotic, a carbapenem-class antibiotic, and a monobactam-class antibiotic. In another embodiment, the antibiotic resistance gene that confers resistance to any other antibiotic agent known in the art. Each possibility may be considered as a separate embodiment of the present invention.

In some embodiments, a PCR reaction of the present invention is an allele-specific PCR (AS-PCR) reaction. AS-PCR is well-known in the art and is described, inter alia, in U.S. Pat. No. 5,496,699.

### Kits

In another embodiment, the present invention provides a kit comprising a PCR reaction mixture of the present invention and instructions for use thereof, for example for amplifying specific target sequences in clinical specimens. In another embodiment, the kit is indicated for detecting a pathogen in a test sample and contains instructions for the detection. In another embodiment, the kit is indicated for forensic or medical application requiring detection of human or animal DNA markers. Each possibility may be considered as being a separate embodiment of the present invention.

Provided herein are reaction mixtures including a set of primers for amplifying a particular species-specific gene, wherein the primers for amplifying at least one end of the target sequence include both hot-start primers and non-hot-start primers. As provided herein, such reaction mixtures exhibit properties superior to mixtures containing either hot-start primers alone or non-hot-start primers alone.

Reference is now made to the following examples, which, together with the above descriptions, illustrate the invention in a non limiting fashion.

Generally, the nomenclature used herein and the laboratory procedures utilized in the present invention include molecular, biochemical, microbiological and recombinant DNA techniques, Such techniques are thoroughly explained in the literature. See, for example, “Molecular Cloning: A Laboratory Manual” Sambrook et al., (2001); “Current Protocols in Molecular Biology” Volumes I-III Ausubel, R. M., ed. (1994); Ausubel et al., “Current Protocols in Molecular Biology”, John Wiley and Sons, Baltimore, Md. (2009); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W.H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds, (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); “Molecular Beacons: Probes that Fluoresce upon Hybridization”, Sanjay Tyagil & Fred Russell Kramer, Nature Biotechnology 14, 303-308 (1996); all of which are incorporated by reference as if fully set forth herein, Other general references are provided throughout this document. The procedures therein are believed to be well known in the art and are provided for the convenience of the reader, All the information contained therein is incorporated herein by reference.

### EXPERIMENTAL DETAILS SECTION

### Materials and Experimental Methods

### Materials

MRSA Strain #1: ATCC700699 (ATCC, Manassas, Va.)

MRSA Strain #2: BAA1556 (ATCC, Manassas, Va.)

MRSA Strain #3: BAA40 (ATCC, Manassas, Va.)

Prep Tube Dolphin tube containing 1 ml of a Sample Preparation Buffer consisting of molecular biology grade water, Tris and Na-EDTA.

Dri-Lyse Tube: 2-ml tube containing stabilized achromopeptidase that is hydrated by a Lysis Buffer consisting of molecular biology-grade water, Tris, and Na-EDTA to form a Lysis Solution.

RNAse H2 enzyme: Pyrococcus abyssi Ribonuclease H2 enzyme was obtained from Integrated DNA Technologies, Inc. (Coralville, Iowa).

### Experimental Methods (Generic to all Examples)

### “Ribo-Primer” Hot-Start Primers

The ribo-primers utilized herein are variants of standard DNA oligonucleotide primers, containing the following modifications: A single RNA residue was added at the 3′-end of standard DNA oligonucleotide primers, followed by four complementary DNA residues and a blocking group that prevents primer extension. The RNA residue renders the primer a substrate for cleavage by RNaseH2 endonuclease at high temperatures, provided that the primer is hybridized to its target. When those conditions have been met, the RNaseH2 endonuclease becomes active and cleaves the resulting heteroduplexes, thereby releasing the blocking modifications and yielding an extendable PCR primer.

qPCR Assay

qPCR was performed as described by Paitan et al (US Application No. 2009/0081663). Multiple primers and dual-labeled Molecular Beacon probes were used for hybridization to three DNA targets found in MRSA. A primer pair and probe for detection of a synthetic DNA were added as an internal control.

### Control DNA

A 344 base pair (bp) synthetic DNA in a plasmid complementary to the internal control primer pair and probe was used to confirm the integrity of the real-time PCR reaction.

### RNaseH2 Mixture

32.5 milli-units/reaction of RNaseH2 endonuclease (Integrated DNA Technologies, Inc.) was diluted in a buffer mixture consisting of Tris, Glycerol, NaCl, EDTA and Triton X100.

### PCR MasterMix

The PCR reaction mixture contained DNA Polymerase (Taq Pol from Jena Bioscience), dNTP's (Jena Bioscience), Tris, KCl, MgCl₂, BSA and sucrose.

### Example 1

### Multiplex PCR Using Hot-Start Primers May Exhibit Amplification Difficulties Under Certain Conditions

### Material and Experimental Methods

### Probes and Primers for Example 1

### Probes

Four dual-labeled Molecular Beacon probes were used, as depicted in Table 1; one complementary to the S. aureus orfX region (SEQ ID NO: 1), a second complementary to the S. aureus-specific nuc gene (SEQ ID NO: 2), a third complementary to the mecA gene (SEQ ID NO: 3), and a fourth complementary to the internal control DNA (SEQ ID NO: 4).

TABLE 1
Dual-labeled Molecular Beacon probes.

| | | 5′-end | 3′-end | Concentration in |
|---|---|---|---|---|
| SEQ ID | Sequence | Fluorophore | Quencher | reaction mixture |
| 1 | cgcgatctcgtcattggcggatc | 6-FAM | BHQ-1 | 0.5 micromolar |
| | aaacggcctgcacgatcgcg | (green) | | |
| 2 | cgcgatcttggttgatacacctg | ROX | BHQ-2 | 0.5 micromolar |
| | aaacaaagcatcctgatcgcg | (orange) | | |
| 3 | cgcgatcctgattcaggttacgg | HEX | BHQ-1 | 0.5 micromolar |
| | acaaggtgatcgcg | (yellow) | | |
| 4 | cgcgatcccaggaagacaggt | Quasar 670 | BHQ-2 | 0.5 micromolar |
| | acaggatcattctgcgatcgcg | (red) | | |

Dual-labeled Molecular Beacon Probes were purchased from Biosearch Technologies Inc. (Novato, Calif.) and incorporate standard fluorophores and quenchers.

### Forward Primers

Four forward primers were used, as depicted in Table 2; one complementary to the S. aureus orfX region (SEQ ID NO: 5), a second complementary to the S. aureus-specific nuc gene (SEQ ID NO: 12), a third complementary to the mecA gene (SEQ ID NO: 14), and a fourth complementary to the internal control DNA (SEQ ID NO: 16). Each primer was a “ribo-primer” hot-start Primer; the ribonucleotide residue in the table is preceded with an “r” and is underlined.

### Reverse Primers

Nine reverse primers, also ribo-primers, were used, as depicted in Table 2: six complementary to different strains of the SCCmec gene (SEQ ID NO: 6-11); a seventh complementary to the S. aureus-specific nuc gene (SEQ ID NO: 13); an eighth complementary to the mecA gene (SEQ ID NO: 15); and a ninth reverse primer used to detect the internal control DNA (SEQ ID NO: 17).

TABLE 2
Forward and reverse primers used in Example 1.
“-▪” represents a blocking group.

| | | Concentration in | |
|---|---|---|---|
| SEQ. ID NO. | Sequence | reaction mixture | |
| SEQ ID NO: 5 | CGCATGACCCAAGGGCA | 0.5 | micromolar |
| | rAAGCG-▪ | | |
| SEQ ID NO: 6 | GTCAAAAATCATGAACC | 0.5 | micromolar |
| | TCATTACTTATGrATAA | | |
| | G-▪ | | |
| SEQ ID NO: 7 | ATTTCATATATGTAATT | 0.5 | micromolar |
| | CCTCCACATCTCrATTA | | |
| | A-▪ | | |
| SEQ ID NO: 8 | CACTTTTTATTCTTCAA | 0.5 | micromolar |
| | AGATTTGAGCrUAAT | | |
| | T-▪ | | |
| SEQ ID NO: 9 | CTCTGCTTTATATTATA | 0.5 | micromolar |
| | AAATTACGGCTGrAAAT | | |
| | A-▪ | | |
| SEQ ID NO: 10 | TGGAAATCCATCTCTAC | 0.5 | micromolar |
| | TTTATTGTTTrUCTT | | |
| | C-▪ | | |
| SEQ ID NO: 11 | TCCATCTCTACTTTATT | 0.5 | micromolar |
| | GTTTTCTTCAArATAT | | |
| | T-▪ | | |
| SEQ ID NO: 12 | AAGCGATTGATGGTGAT | 0.35 | micromolar |
| | ACGrGTTAA-▪ | | |
| SEQ ID NO: 13 | AAATGCACTTGCTTCAG | 0.35 | micromolar |
| | GACrCATAT-▪ | | |
| SEQ ID NO: 14 | GGTGAAGATATACCAAG | 0.35 | micromolar |
| | TGATTArUCCAT-▪ | | |
| SEQ ID NO: 15 | GTGAGGTGCGTTAATAT | 0.35 | micromolar |
| | TGCrCATTA-▪ | | |
| SEQ ID NO: 16 | GCCTCAGTATGCTCCCA | 0.15 | micromolar |
| | CGACCrGAATT-▪ | | |
| SEQ ID NO: 17 | TCAGCAGTTTGCACATC | 0.15 | micromolar |
| | AGGCGrAATAA-▪ | | |

### Experimental Setup

An assay mixture containing the 13 “ribo-primer” hot-start Primers, the 4 Molecular Beacon Probes, the Internal Control DNA and the PCR MasterMix was prepared and hydration-reduced to enhance stability by heating at 55° C. for 2 hours.

Immediately prior to use, the mixture was hydrated by 25 μl of a solution containing the RNaseH2 Mixture and purified DNA from culture samples of MRSA, and a multiplex qPCR reaction was performed.

The assay was tested with two separate strains of MRSA (MRSA Strain #1 and MRSA Strain #2), each performed in duplicate. For Example #1, DNA from each MRSA strain was purified using the DNeasy™ Blood & Tissue Kit (QIAGEN GmbH), and a total of 0.01 nanograms of purified DNA (equal to ˜300 CFU of bacteria) was used for each reaction. A negative control sample was included in the run, containing the assay mixture in TE and no template DNA.

Amplification and detection reactions were run using a RotorGene 6000 System Real

Time PCR instrument from Corbett Life Science according to the following standard qPCR protocol:
1. 3 minutes at 95° C. to denature the DNA.
2. amplification cycles each consisting of the following three steps: (a) 20 seconds at 95° C.; (b) 60 seconds at 56° C.; (c) 30 seconds at 72° C. (at the end of step (b), the readings were taken for each of the four fluorescent dyes).

### Results

A multiplex qPCR assay for detecting Methicillin-Resistant Staphylococcus aureus (MRSA) was performed. This assay detects three different sequences: the mecA gene, which is associated with methicillin-resistance (“yellow channel”); the S. aureus-specific nuc gene (“orange channel”); and SCCmec:orfX, the region between the SCCmec insertion cassette and the orfX region of the S. aureus genome, also referred to as the “bridging region” (“green channel”). The assay utilizes multiple primers and a dual-labeled probe for hybridization of the right extremity junction of SCCmec:orfX, since this region exhibits sequence variation among different S. aureus strains. A primer pair and probe for detection of a synthetic DNA were included as an internal control (“red channel”), as well as a negative control with no template DNA. The assay was conducted with purified DNA extracted from culture samples of two separate strains of MRSA, MRSA strains #1 and #2, each performed in duplicate. The nuc primers recognize sequence specific for S. aureus nue; thus they do not amplify S. epidermis.

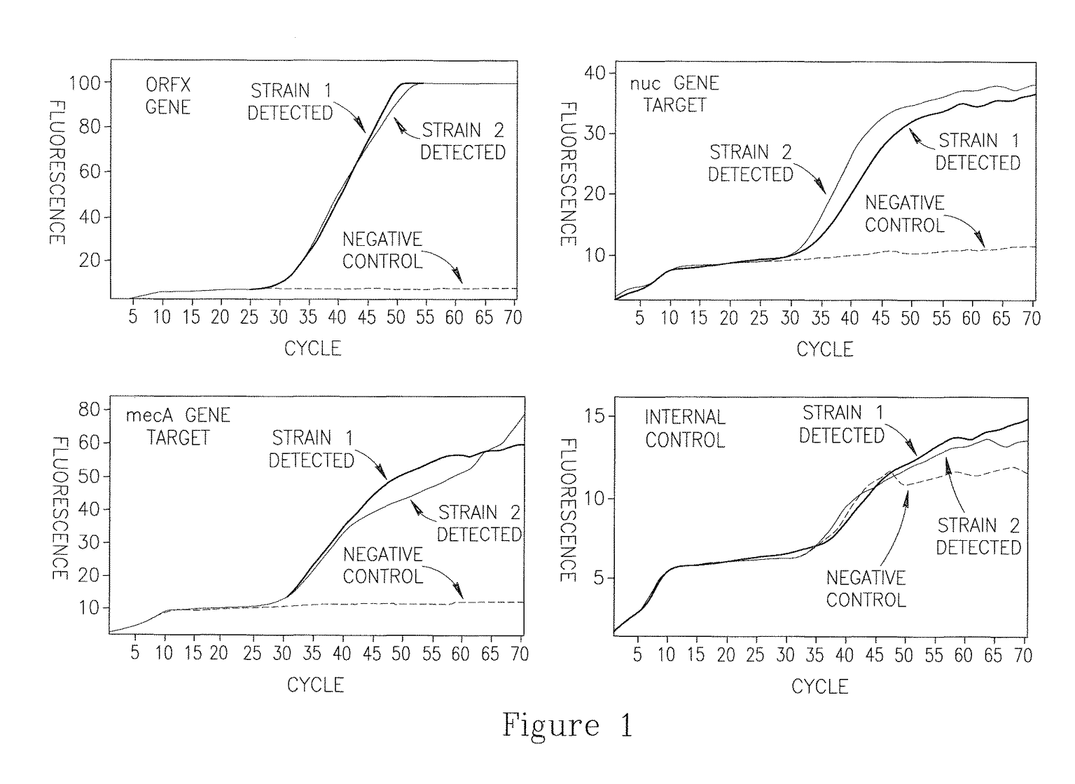

As presented in FIG. 1, the probes for the internal control (IC), orfX and mecA gene each produced positive signals that were nearly identical across the two MRSA strains. The nuc gene targeting probe produced a positive signal, but exhibited a noticeable difference between the two strains, in that the assay amplified DNA from Strain #1 noticeably later than Strain #2. Duplicate samples exhibited high reproducibility.

Examination of the published sequences of the two strains revealed that Strain #2 forms a perfect complement to both the forward and reverse nuc primers used in the detection assay, while Strain #1 contains a single mismatched base positioned one base prior to the RNA base of the reverse nue primer. In summary, while the reaction mixture containing hot-start primers was able to amplify all DNA targets in the four-plex assay, the one DNA target that contained a mismatched base exhibited noticeably less robust amplification than a DNA target containing no mismatched bases.

### Example 2

### Clinical Nasal Swab Samples May Inhibit PCR Using Hot-Start Primers

### Experimental Methods

### Preparation of the First Sample Type (“Spiked Nasal Sample”)

1. A monodisperse solution was prepared from each bacterial strain in TE pH 8.3, using the Detect-Ready™ MRSA Lysis Kit as follows:
   a. A single colony was picked with a sterile bacteriological loop and shaken into 1 ml TE pH 8.3 Buffer.
   b. Dilutions were made to reach a concentration of 10⁴ cfu per ml, by transferring 100 μl into 900 μl of TE (Tris-EDTA), then mixing and repeating this step two additional times.
2. With a sterile pipette tip, 10 μl of the diluted bacterial colony (equivalent ˜300 cfu) was removed and spiked onto a saline-moistened nasal swab that was swabbed inside a nostril of a human volunteer.
3. A Prep Tube was opened and the swab inserted until the head of the swab was fully submerged in the Sample Preparation Buffer solution.
4. The swab was agitated in the buffer for 10-15 seconds (vigorously, but with caution, to avoid spilling the tube contents) to release the bacteria.
5. The swab was discarded, and the Prep Tube was closed tightly.
6. The Prep Tubes were placed in a centrifuge and centrifuged at 10,000-12,000 g for 5 minutes at room temperature.
7. During the centrifugation step, the Dri-Lyse tubes were rehydrated by adding 1 ml of Lysis Buffer. The Dri-Lyse Tubes were closed and vortexed for 5 seconds to reconstitute a Lysis Solution,
8. The Prep Tubes were removed from the centrifuge and the supernatant poured out by gently inverting the tube.
9. Without touching the pellet attached to the tube bottom, residual buffer was removed by gentle aspiration with a 200 μl micropipette while holding the tube in a horizontal position with the pellet oriented upwards.
10. 90 μl of Lysis Solution was added to each pellet-containing Prep Tube.
11, Pellets were detached from the bottom of the tubes by pipette.
12. The Prep Tubes were incubated at 37° C. for 10 minutes in a heating block, heated at 99° C. for 5 minutes, and removed from the heating block.
13. The samples were centrifuged for 1 min at maximum speed.
14. 10 μl of the RNaseH2 solution Mixture was added to each sample containing Prep Tube.
15. 25 μl of the solution in each Prep Tube was then transferred into a PCR Reaction. Tube containing the qPCR reaction mixture described above in Example 1 and a multiplex qPCR reaction was run as described in Example 1.

### Preparation of the Second Sample Type (“Direct Sample”)

10 μl of the diluted bacterial colony (equivalent to ˜300 cfu) was removed and pipetted directly into a rehydrated Dri-Lyse Tube containing 90 μl of liquid. The tube was then incubated at 37° C. for 10 minutes in a heating block, heated at 99° C. for 5 minutes, and removed from the heating block. 10 μl of the RNaseH2 Mixture was added and then 25 μl of the resultant solution was then transferred into a PCR Reaction Tube containing the qPCR reaction mixture described above in Example 1. A multiplex qPCR reaction was run as described in Example 1.

### Results

A multiplex qPCR assay for detecting MRSA was performed using the same reaction mixture and hot-start primers as described for Example 1, in this case targeting two types of DNA samples that had been obtained without extensive purification (using the Detect-Ready™ MRSA Lysis Kit) from each of the two MRSA bacterial strains tested in Example 1. The first type of sample (“Spiked Nasal sample”) had been spiked onto clinical nasal sample swabs, while the second type (“Direct sample”) had not.

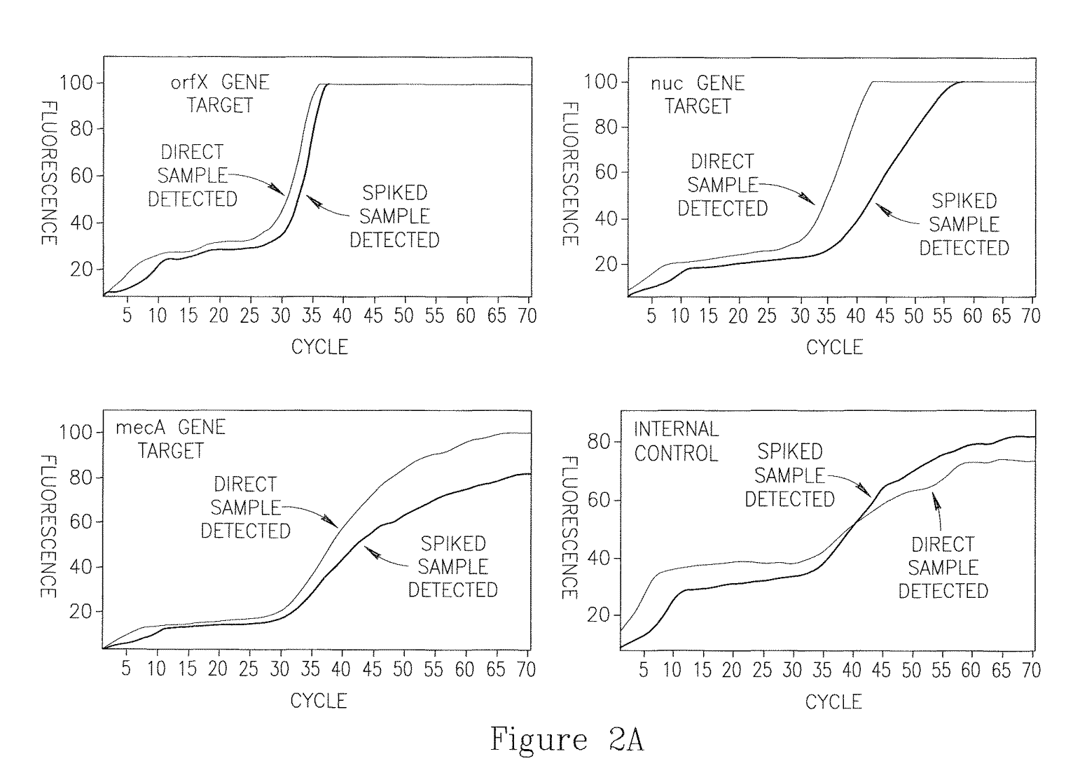

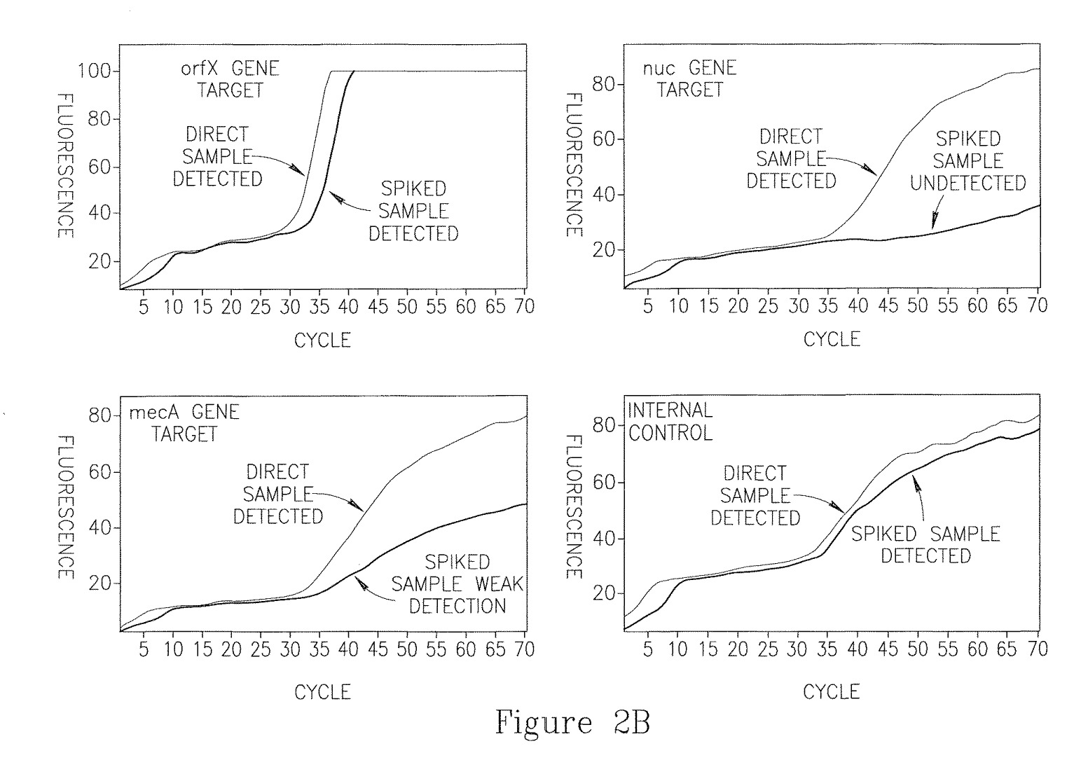

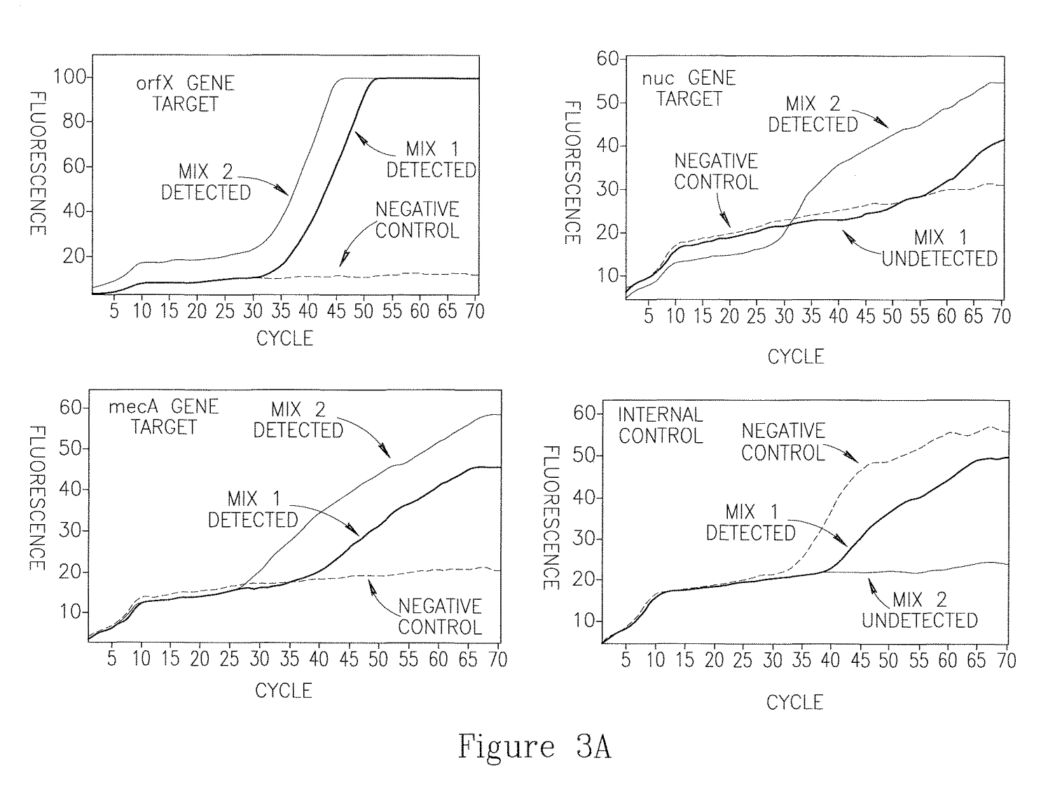

For MRSA Strain #2, all four probes produced a positive signal in the case of both the nasal and Direct samples; however, the Spiked Nasal samples exhibited systematically higher cycle numbers, indicating delayed amplification (FIG. 2A). This was also true for IC, mecA and OrfX in the case of MRSA Strain #1. However, amplification of the nuc gene of MRSA Strain #1 was completely inhibited in the Spiked Nasal samples (FIG. 2B). Duplicate samples exhibited high reproducibility. Over 20 strategies were attempted to overcome this inhibition, the vast majority of which had no effect on inhibition and/or a detrimental effect on assay sensitivity (data not shown).

These results show that the Spiked Nasal samples contained a component that interfered with optimal performance of the PCR reactions, in the case of a perfect primer-target match, and completely inhibited the reaction when a primer-target mismatch was present.

### Example 3

### A Nested Combination of Hot-Start Primers and Regular Primers Overcomes Inhibition of qPCR by Nasal Swab Samples

### Experimental Methods

### Primers

The non-hot-start forward and reverse primers used in Example 3 are depicted in Table 3 and are described below.

| SEQ ID/ | | Concentration in | |
|---|---|---|---|
| identity | Sequence | reaction mixture | |
| SEQ ID No: 18 | CGCATGACCCAAGGG | 0.05 | micromolar |
| orfX forward | CA | | |
| SEQ ID No: 19 | ATTTCATATATGTAA | 0.05 | micromolar |
| SCCmec rev | TTCCTCCACATCTC | | |
| SEQ ID No: 20 | GTCAAAAATCATGAA | 0.05 | micromolar |
| SCCmec rev | CCTCATTACTTATG | | |
| SEQ ID No: 21 | CTCTGCTTTATATTA | 0.05 | micromolar |
| SCCmcc rev | TAAAATTACGGCTG | | |
| SEQ ID No: 22 | CACTTTTTATTCTTC | 0.05 | micromolar |
| SCCmec rev | AAAGATTTGAGC | | |
| SEQ ID No: 23 | TGGAAATCCATCTCT | 0.05 | micromolar |
| SCCmec rev | ACTTTATTGTTT | | |
| SEQ ID No: 24 | TCCATCTCTACTTTA | 0.05 | micromolar |
| SCCmec rev | TTGTTTTCTTCAA | | |
| SEQ ID No: 25 | AAGCGATTGATGGTG | 0.035 | micromolar |
| nuc for | ATACG | | |
| SEQ ID No: 26 | AAATGCACTTGCTTC | 0.035 | micromolar |
| nuc rev | AGGAC | | |
| SEQ ID No: 27 | GGTGAAGATATACCA | 0.035 | micromolar |
| mecA for | AGTGATTA | | |
| SEQ ID No: 28 | GTGAGGTGCGTTAAT | 0.035 | micromolar |
| mecA rev | ATTGC | | |

### Experimental Protocol

Two types of non-extensively purified DNA samples were prepared as described for Example 2, in this case using MRSA Strain #3. A multiplex qPCR assay for detecting MRSA was performed using several different reaction mixtures as follows:

Mixture #3-1: the same reaction mixture, containing hot-start primers only, as described for Examples 1-2.

Mixture #3-2: a mixture of the same probes and 13 hot-start primers as in the previous mixture with 11 additional regular (non-hot-start) unmodified oligonucleotide primers for nuc, orfX and mecA (SEQ 18-28), each present at 10% of the molar quantity of its corresponding hot-start primer, forming a nested primer design. The sequence of each regular primer was somewhat longer than the corresponding hot-start primer and was complementary to a region 1-70 bases outside of the region targeted by the hot-start primer. The Internal Control assay utilized hot-start primers only.

Mixture #3-3: Non-hot-start nested primers only at 10% of the usual concentration (100% of the same concentration as the regular primers in Mixture #2) for orfX, nuc, and mecA; hot-start primers only for IC.

### Results

Spiked Nasal samples and Direct samples were prepared from non-extensively purified DNA, as described for Example 2, in this ease using MRSA Strain #3. MRSA Strain #3 was chosen because qPCR reactions using hot-start primers for both the nuc targeting assay and the mecA targeting assay had been found to be substantially impaired when applied to clinical nasal swab samples containing Strain #3. Multiplex qRT-PCR assays were performed using the 3 mixtures described in the Methods section of this Example. Duplicate samples exhibited high reproducibility.

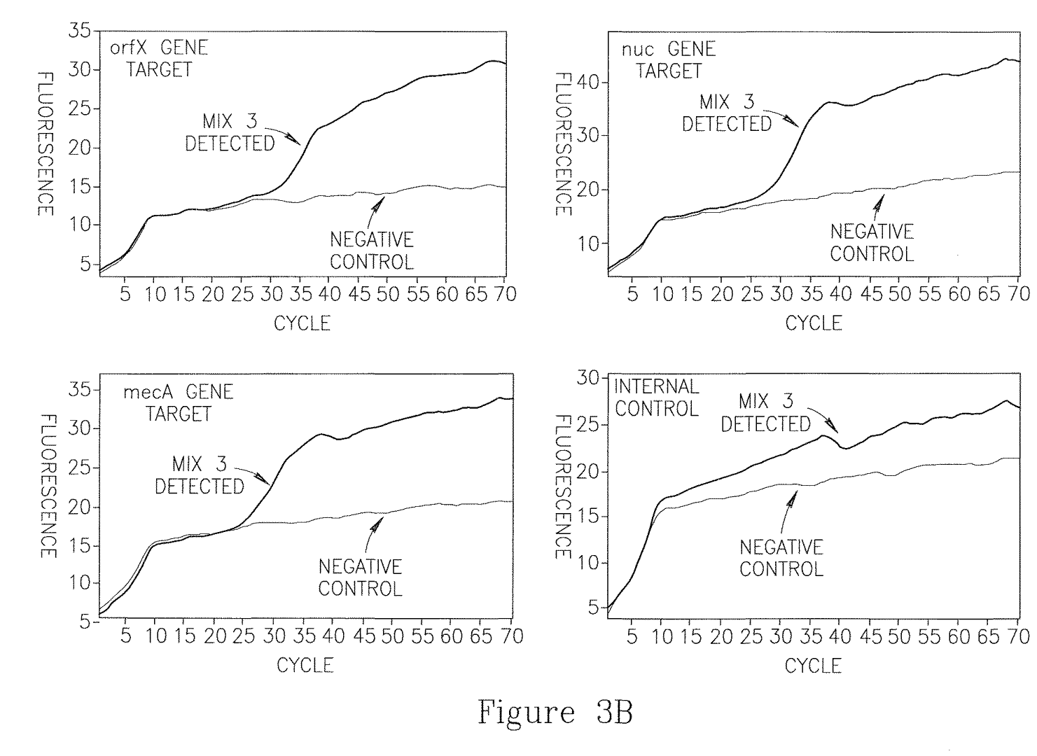

For Mixture 43-1, as in the previous Example, IC and OrfX were efficiently amplified in the case of both the Spiked Nasal samples and Direct samples, similar to the results seen with MRSA Strains #1 and #2 in Example 2. The results with mecA and nuc varied depending on the sample type. In the Direct samples, these regions were efficiently amplified (not shown), while in the Spiked Nasal samples, amplification of mecA and nuc were considerably inhibited and completely inhibited, respectively (FIG. 3A). These results confirm that the PCR inhibitory effect of clinical specimens is more pronounced in assays utilizing only hot-start primers and varies in its magnitude, depending upon the extent of primer-target hybridization. In this experiment, for example, the inhibiting effect was minimal in the case of the orfX, more substantial for mecA, and nearly total for the nue gene targeting assay. The negative control for Mixture #3-1 is also shown; this control exhibited a higher signal on the internal control channel due to relatively low competition from competing channels.

For Mixture #3-2, by contrast (also shown in FIG. 3A), robust amplification was seen on all three experimental channels in the Spiked Nasal samples. These results demonstrate that utilization of a mixture of hot-start—and non-hot-start primers, even including as little as 10% non-hot-start primers, provides a booster effect that largely overcomes the PCR inhibitory effect of clinical specimens.

For Mixture #3-3, all three amplifications were successful in the Spiked Nasal samples to the extent that the respective DNA targets were detected (FIG. 3B). However, comparison of these results to a mixture of hot-start-primers and nested regular primers (Mixture #3-2; FIG. 3A) indicates that Mixture #3-2 achieved substantially stronger amplification, even in the presence of PCR inhibition. In the latter case, the amplification driven by the non-hot-start primers reached a plateau, and the hot-start primers continued from that time point forward to direct further amplification. The negative control for Mixture #3-3 is also shown.

Thus, the nested combination of hot-start primers and regular primers (Mixture #3-2) overcame the inhibition of hot-start primer PCR observed in the presence of clinical specimens (Mixture #3-1) and was also superior to use of a combination of hot-start primers only for target exhibiting reduced hybridization and non-hot-start primers only for uninhibited targets (Mixture #3-3).

### Example 4

### qPCR Reactions Utilizing Hot-Start Primers are Inhibited by Parallel Amplifications Using Regular Primers

This experiment also utilized purified MRSA Strain #3 DNA samples, present as either Spiked Nasal and Direct samples not subject to extensive purification after spiking, as described for Example 3. In this case, only hot-start primers were used for the assays targeting IC and OrfX (i.e. those shown earlier to achieve strong PCR amplification even in the presence of PCR inhibition from clinical swab samples); while regular unmodified primers having sequences identical to those used for the hot-start primers used in Examples 1-2 were utilized for nuc and mecA (which had exhibited extensive PCR inhibition in the presence of clinical swab samples). Spiked Nasal samples were run in duplicates, and a negative control sample was included. An additional control reaction was also performed, utilizing hot-start primers alone for all reactions.

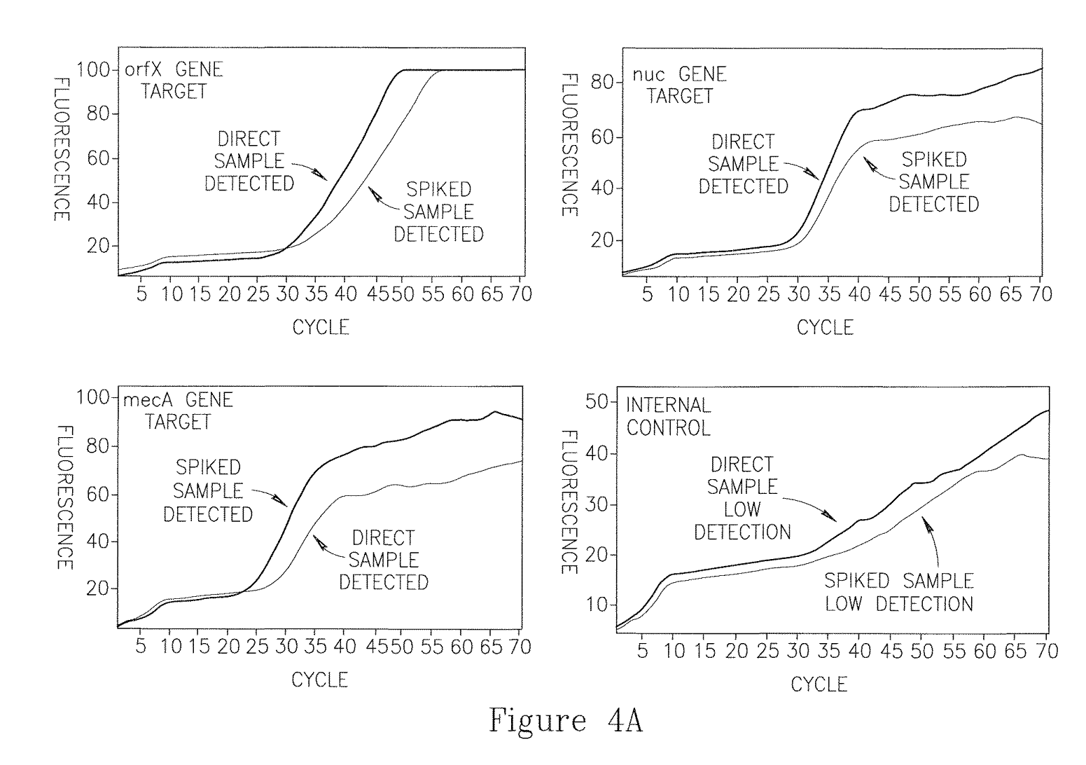

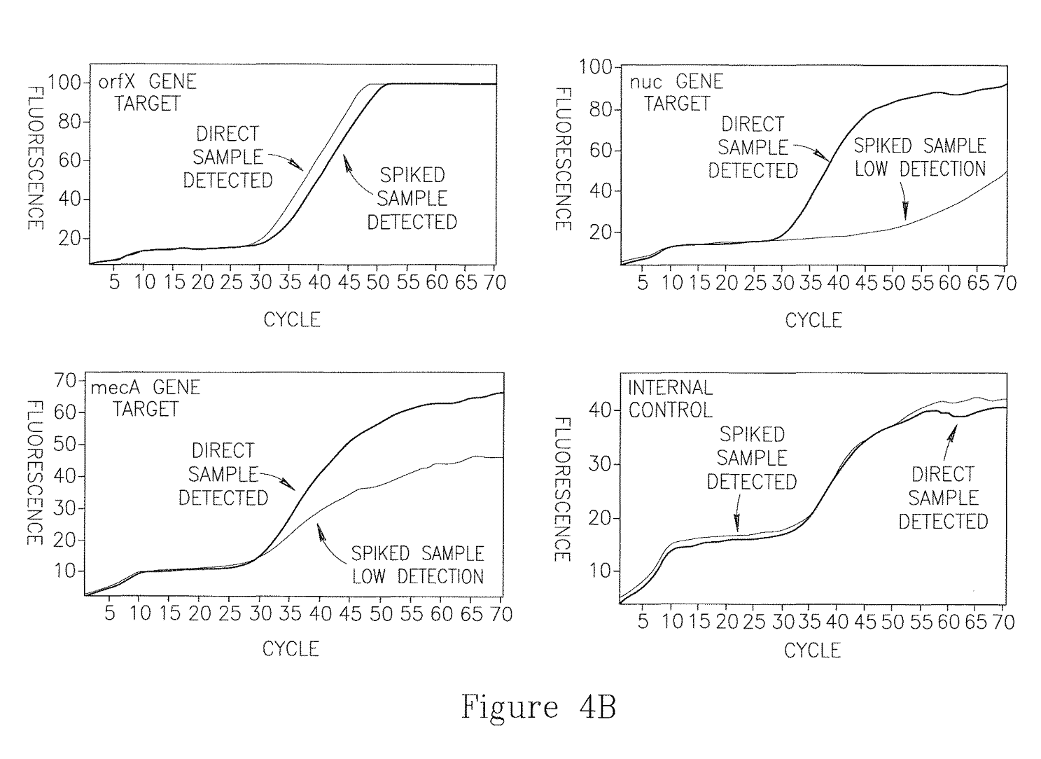

In the control reaction containing hot-start primers alone, orfX and IC were amplified, while detection of nuc and mecA was inhibited, as observed previously (FIG. 4B). Amplifications utilizing regular primers (nuc and mecA) were found to perform well; mecA, in particular, exhibited stronger amplification than in previous Examples (FIG. 4A). The Spiked Nasal sample used in this Example was found upon separate examination to be from an individual known to have nasal carriage of methicillin-resistant Staphylococcus Epidermis bacteria, which likely accounted for the stronger amplification of mecA gene. While orfX and IC were amplified in both samples, amplification of the Spiked Nasal sample was somewhat delayed by the use of the regular primers in the other channels, as shown by a comparison to the hot-start only control reaction (FIG. 4C). Duplicate samples exhibited high reproducibility.

In an attempt to reduce inhibition of mecA and OrfX, the concentrations of regular primers targeting nuc and mecA were reduced. This strategy did not appreciably improve amplification of the IC (data not shown).

Thus, amplifications utilizing only hot-start primers are likely to be inhibited in the context of a multiplex assay in the presence of other amplifications utilizing only regular primers. Without wishing to be bound by any particular theory or mechanism of action, the inhibition of the hot-start amplifications in this case is likely to be due to competition for resources such as dNTPs in the reaction tube.

### Example 5

### Use of a Mixture of Hot-Start and Regular Primers Prevents Inhibition of Competing Amplifications in Multiplex qPCR

### Primers

The non-hot-start (regular) forward and reverse primers used in Example 5 are depicted in Table 4 and are described below.

TABLE 4

| SEQ | |
|---|---|
| ID | Sequence |
| 29 | GAACCAACGCATGACCCAAGGGCAAAGCG |
| 30 | TCCAATATTTCATATATGTAATTCCTCCACATCTCATTAA |
| 31 | AACTATGTCAAAAATCATGAACCTCATTACTTATGATAAG |
| 32 | AATAAACTCTGCTTTATATTATAAAATTACGGCTGAAATA |
| 33 | AATATTCACTTTTTATTCTTCAAAGATTTGAGCTAATT |
| 34 | GAGGATATGGAAATCCATCTCTACTTTATTGTTTTCTTC |
| 35 | TGGAAATCCATCTCTACTTTATTGTTTTCTTCAAATATT |
| 36 | GGTGTTGGTGAAGATATACCAAGTGATTATCCAT |
| 37 | TAATAAGTGAGGTGCGTTAATATTGCCATTA |
| 38 | TAATTAAAGCGATTGATGGTGATACGGTTAA |
| 39 | TTTCGTAAATGCACTTGCTTCAGGACCATAT |

In this experiment, three different primer mixes were utilized for each of the assays targeting the orfX, nuc and mecA genes:

Mixture #5-1 contained a mixture of hot-start and non-hot-start (regular) primers. With each hot-start primer, a regular primer was included, having a sequence identical to the hot-start primer, but with the addition of additional complementary bases at either end to provide preferential binding, as depicted in Table 5. The concentration of primers were the same as in the assay mixtures described for Examples 1 and 2, except that they contained 80% hot-start primers and 20% regular primers. Mixture #5-2 was a control mixture containing no hot-start primers and the same amount of regular primers as the amount of regular primers in Mixture #5-1.

Mixture #5-3 contained hot-start primers only, at the concentration levels presented above in Examples 1 and 2.

Hot-start primers were used exclusively in all mixtures for amplification of the IC, as in Example 4. Non-extensively purified DNA was prepared from bacteria spiked onto nasal samples and direct samples of MRSA Strain #3, as described for Example 3. Nasal samples were run in duplicates, and a negative control sample was included.

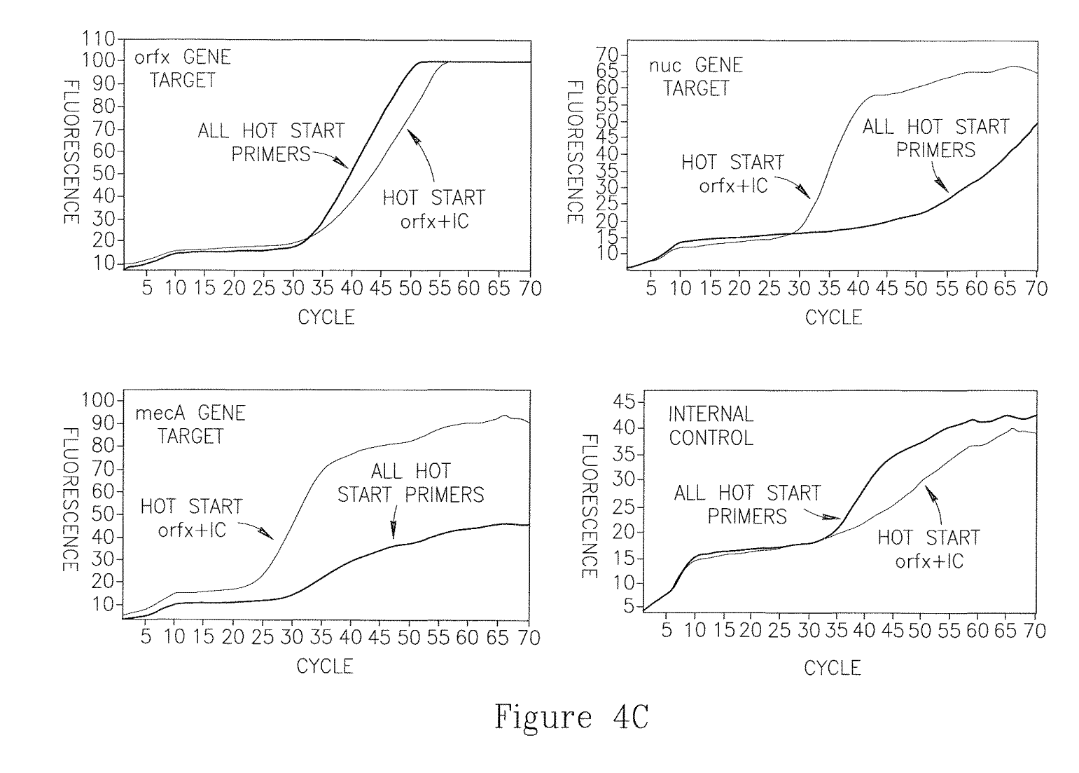

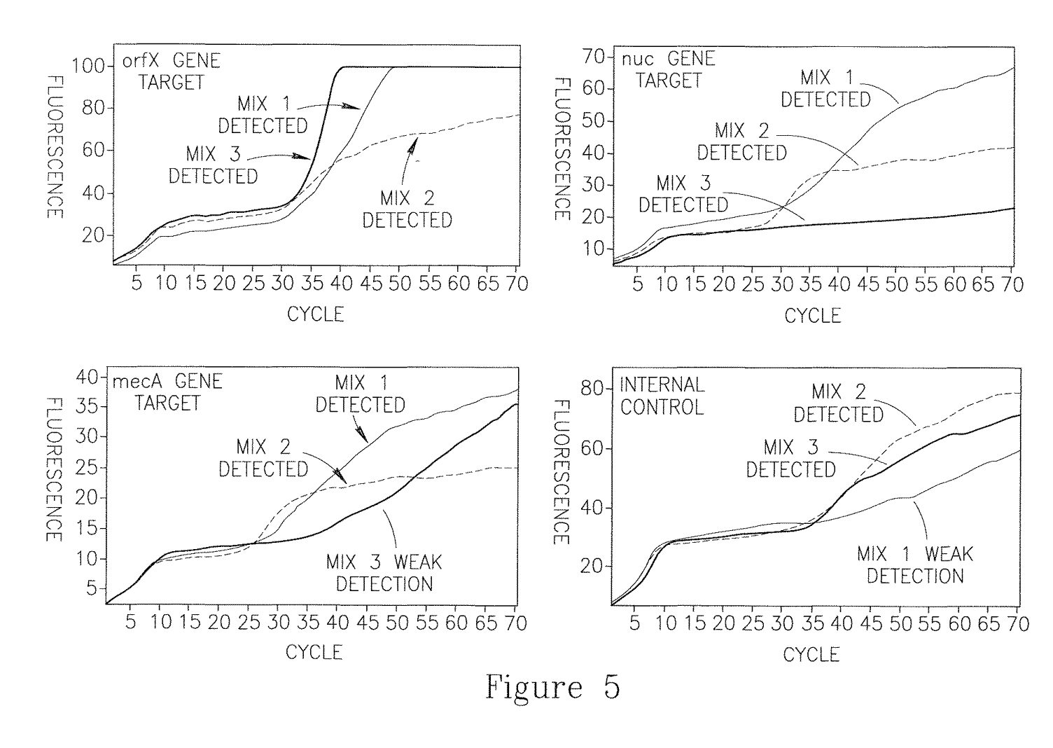

Positive amplification was evident in all four channels in the samples utilizing a mixture of hot-start primers and regular primers (FIG. 5, Mixture #1). These results were a significant improvement over use of hot-start primers alone for all reactions (Mixture #3), or use of only hot-start primers for certain reactions and use of only regular primers for other reactions (Example 4). This Example thus demonstrates an effective strategy for performing multiplex qPCR reactions on clinical specimens likely to contain inhibitors, and/or reactions wherein some amplifications are expected to exhibit weak primer-target hybridization, as in the case of a primer-target mismatch, and other amplifications are expected to exhibit strong primer-target hybridization. In addition, this Example demonstrates that in cases where a PCR reaction using hot-start primers alone is inhibited (FIG. 5, nue gene target, Mixture #3), the inclusion of corresponding regular primers provides a boost enabling the hot-start primers to amplify the target (FIG. 5, nuc gene target, compare Mixture #2 to Mixture #1). Without wishing to be bound by any particular theory or mechanism of action, the improved balance between the boosting effect of the nuc and mecA amplifications and the preserved efficacy of the orfX and IC amplifications in this case is likely to be due to binding competition for target sequences between the hot-start and non-hot-start primers.

### Example 6

### Superior Results Obtained by Varying the Concentration of Primers Used in the Previous Example

The experiment of the previous Example was repeated, with the concentrations of primers for the assays targeting orfX and IC identical to the previous Examples, and the nuc and mecA primer concentrations adjusted to optimize performance of the 4 distinct assays in the multiplex assay reaction mixture as follows (Table 5):

TABLE 5
Primer concentrations used in Examples 6.

| Target | Hot-start primer conc. (mM) | Regular primer conc. (mM) |
|---|---|---|
| Nuc | 0.105 × 2 = 0.210 | 0.175 × 2 = 0.350 |
| mecA | 0.105 × 2 = 0.210 | 0.245 × 2 = 0.490 |
| OrfX region | 0.5 × 7 = 3.5 | 0 |
| IC | 0.15 × 2 = 0.3 | 0 |

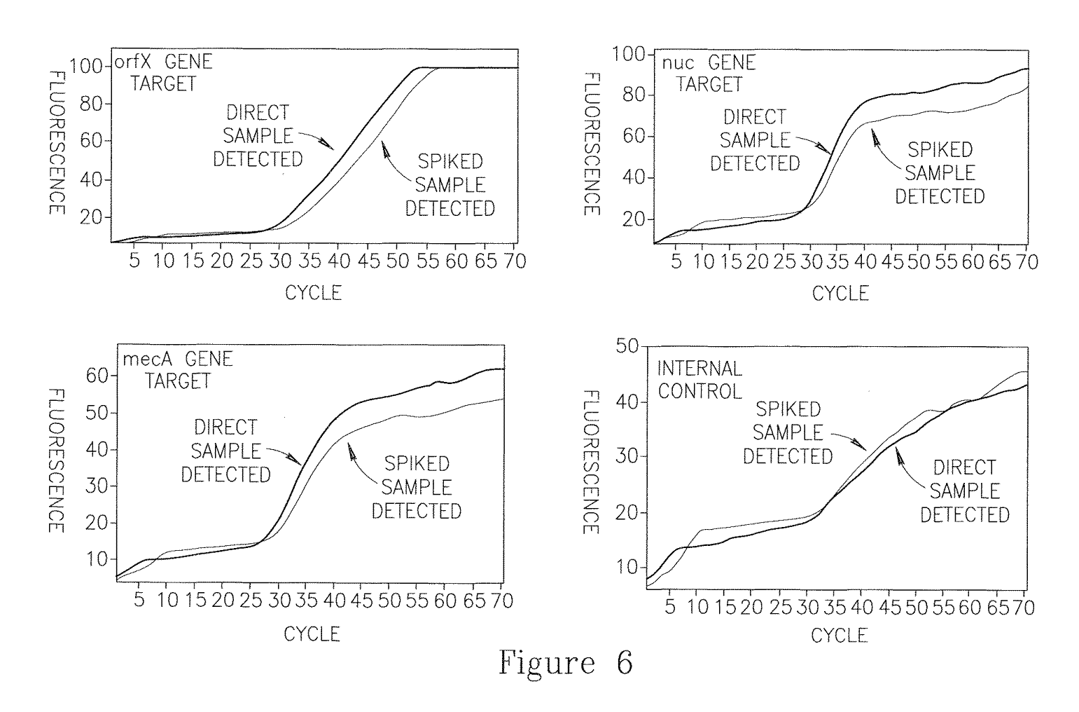

The orfX/SCCmec region was amplified using a 0.5 mM micromolar concentration (0.5 mM) of each of the 7 hot-start primers. The nuc gene region was amplified using 0.105 mM of each of the 2 hot-start primers and 0.175 mM of each of the 2 non-hot-start primers. The mecA gene region was amplified using 0.105 mM of each of the 2 hot-start primers and 0.245 mM of each of the 2 non-hot-start primers. The internal control was amplified using 0.15 mM of each of the 2 hot-start primers. Readily quantifiable amplification was obtained in all four channels (FIG. 6).

This and the previous Example thus present another effective strategy for performing multiplex qPCR reactions on clinical specimens likely to contain inhibitors, wherein some amplifications exhibit weak primer-target hybridization, as in the case of a primer-target mismatch, and other amplifications exhibit strong primer-target hybridization. A mixture of hot-start and regular primers is utilized for amplification(s) where a mismatch or other weak primer-target hybridization is likely 1.5 to be present, and hot-start primers only are utilized for the amplification(s) where a strong primer-target hybridization is likely to be present.

### Example 7

### Multiplex qPCR Assay Using Hot-Start Primers and Non-Hot Start Primers and Targeting DNA Extracted from Bacterial Cultures Spiked into Clinical Stool Samples

### Bacterial Targets

Clostridium difficile Strain: ATCC 43598 (ATCC, Manassas, Va.)

Escherichia coli (O157) Strain: ATCC 700728 (ATCC, Manassas, Va.)

Staphylococcus Strain: BAA1556 (ATCC, Manassas, Va.)

### Clinical Stool Samples

Frozen residual negative clinical stool samples are obtained from the Hadassah Medical Center (Jenisalem, Israel).

### Sample Preparation

1. A monodisperse solution is prepared from the bacterial strain in buffered solution as follows:
   a. A single colony is picked with a sterile bacteriological loop and shaken into buffer.
   b. Serial dilutions are performed to reach an appropriate concentration,
2. With a sterile pipette tip, an aliquot of the solution of bacteria is removed and spiked onto a clinical swab from a stool sample.
3. The supernatant is then pipetted into a Prep Tube, which is closed and centrifuged to reduce the level of particulate matter.
4. The supernatant is decanted, and residual buffer is removed.
5. Pellets are dissolved in lysis buffer.
6. Prep tubes are incubated on a heating block, then centrifuged.
7. RNaseH2 solution mixture is added to each Prep Tube.
8. An aliquot of the solution in each Prep Tube is then transferred into a PCR Reaction Tube containing a qPCR reaction mixture.

### Hot-Start and Non-Hot Start Primers

Two versions of each primer are designed. The first version is a hot-start primer, using the ribo-primer structure described in earlier Examples. The second version is a non-hot-start unmodified primer structure that does not include the added RNA base, four complementary bases, and blocking group at the 3′-end.

### Probes

Three dual-labeled Molecular Beacon probes (Biosearch Technologies Inc, Novato, Calif.) are used, complementary to C. Difficile tcdB (SEQ ID NO: 40), E. coli eae (SEQ ID NO: 41), and Staphylococcus tuf (SEQ ID NO: 42), respectively (Table 6).

TABLE 6
Dual-labeled Molecular Beacon probes. Stem-forming
residues are depicted in CAPS.

| | | 5′-end | 3′-end | Concentration in |
|---|---|---|---|---|
| SEQ ID | Sequence | Fluorophore | Quencher | reaction mixture |
| 40 | CGCGCTCcattaacattata | 6-FAM | BHQ-1 | 0.25 micromolar |
| | gtcactatttacattttccatt | (green) | | |
| | gaGAGCGCG | | | |
| 41 | CGCAGGgccgacctgacc | ROX | BHQ-2 | 0.25 micromolar |
| | aaatgccagcatcCCTGCG | (orange) | | |
| 42 | CGCGCCTggacgtattctc | Quasar 670 | BHQ-2 | 0.25 micromolar |
| | aatcactggtcgtggtactgAG | (red) | | |
| | GCGCG | | | |

### Forward Primers

Three sets of ribo- and unmodified forward primers are used, as depicted in Tables 7-8, respectively; complementary to Ted B (SEQ ID NO: 43 and 49), ear (SEQ ID NO: 45 and 51), and tuf (SEQ ID NO: 47 and 53).

### Reverse Primers

Three sets of ribo- and unmodified reverse primers are used, as depicted in Tables 7-8, respectively: complementary to Ted B (SEQ ID NO: 44 and 50), eae (SEQ ID NO: 46 and 52), and tuf (SEQ ID NO: 48 and 54).

TABLE 7
Forward and reverse hot-start primers used in
Example 7. “-▪” represents a blocking group.

| | | Concentration in |
|---|---|---|
| SEQ ID NO. | Sequence | reaction mixture |
| SEQ ID NO: 43 | GGATTGGAGGTCAAATAAA | 0.25 micromolar |
| | TGArCACTG-▪ | |
| SEQ ID NO: 44 | TTTCAATGTGTTTATCAAA | 0.25 micromolar |
| | AATGCATTArCTATC-▪ | |
| SEQ ID NO: 45 | TAAAGCGGGAGTCAATGrT | 0.25 micromolar |
| | AACG-▪ | |
| SEQ ID NO: 46 | CACAGTTGCAGGCCTGrGT | 0.25 micromolar |
| | TAC-▪ | |
| SEQ ID NO: 47 | TCTGACAAACCATTCATGA | 0.25 micromolar |
| | TrGCCAG-▪ | |
| SEQ ID NO: 48 | GCTTCAGCGTAGTCTAATA | 0.25 micromolar |
| | ATTTACrGGAAC-▪ | |

TABLE 8
Forward and reverse non-hot-start primers used
in Example 7

| | | Concentration in |
|---|---|---|
| SEQ ID | Sequence | reaction mixture |
| SEQ ID No: 49 | ggattggaggtcaaataaat | 0.05 micromolar |
| | gacactg | |
| SEQ ID No: 50 | tttcaatgtgtttatcaaaa | 0.05 micromolar |
| | atgcattactatc | |
| SEQ ID No: 51 | taaagcgggagtcaatgtaa | 0.05 micromolar |
| | cg | |
| SEQ ID No: 52 | cacagttgcaggcaggttac | 0.05 micromolar |
| SEQ ID No: 53 | tctgacaaaccattcatgat | 0.05 micromolar |
| | gccag | |
| SEQ ID No: 54 | gatcagcgtagtctaataat | 0.05 micromolar |
| | ttacggaac | |

RNaseH2 Mixture Pyrococcus abyssi RNaseH2 endonuclease is obtained from Integrated DNA Technologies, Inc.

### PCR MasterMix

A PCR reaction mixture containing Tag Pol DNA Polymerase, dNTP's, Tris, KCl, MgCl₂, BSA and sucrose is utilized,

qPCR Assay

Three distinct assay mixtures, each including the 3 Molecular Beacon Probes, the PCR MasterMix, and a distinct set of primers, are prepared. The primers in the first assay mixture are the 6 “ribo-primer” hot-start primers. The primers in the second assay mixture are the 6 non-hot-start primers. The third assay mixture includes a mixture of the 6 hot-start primers and the 6 non-hot-start primers.

### Procedure

Multiplex qPCR reactions are performed using each of the assay mixtures. A negative control sample is included in the run, containing the third multiplex assay mixture in TE and no template DNA.

Amplification and detection reactions are run using a RotorGene 6000 System Real Time PCR instrument from Corbett Life Science:
1. 3 minutes at 95° C. to denature the DNA.
2. amplification cycles each consisting of the following three steps: (a) 20 seconds at 95° C.; (b) 60 seconds at 56° C.; (c) 30 seconds at 72° C. (at the end of step (b), the readings are taken for each of the three fluorescent dyes).

### Results

Multiplex qPCR assays for detecting toxic Clostridium Difficile, pathogenic Escherichia coli and Staphylococcus are performed. Each multiplex assay is designed to detect the same three distinct sequences: (a) the tcd B gene, which is associated with toxic Clostridium Difficile; (b) the eae gene, which is associated with pathogenic Escherichia coli and (c) the Staphylococcus specific tuf gene. Use of a mixture of hot-start and unmodified primers enables efficient amplification of all targets, even in clinical specimens found to inhibit certain hot-start PCR reactions

### Example 8

### Multiplex qPCR Assay Using Hot-Start Primers Targeting Non-Purified DNA Extracted from Culture Samples Spiked into Various Clinical Specimens

### Materials and Experimental Methods

### Experimental Method

A multiplex qPCR assay is performed using a reaction mixture containing only hot-start primers, similar to Example 1, and using the same qPCR protocol as in previous Examples or a similar protocol to detect DNA extracted from MRSA strain #3. One sample is prepared following Protocol 8-1, and is compared with a sample prepared for qPCR analysis using one the following methods:
The MRSA strain is spiked onto a clinical swab from a throat culture sample, and the sample is prepared following Protocol 8-2.
The MRSA strain is spiked onto a clinical swab from a stool sample, and the sample is diluted in a buffer solution, centrifuged at a low speed to reduce the level of particulate matter in the sample, and the sample is prepared following Protocol 8-2.
The MRSA strain is spiked onto a clinical swab from a sample of vaginal secretion, cerebrospinal fluid, urine, lymph fluids, tears, saliva, milk, amniotic fluid, or an external secretion of the respiratory, intestinal or genitourinary tract, and the sample is diluted in a buffer solution, centrifuged at a low speed to reduce the level of particulate matter in the sample, and prepared following Protocol 8-2.
The MRSA strain is spiked onto a clinical swab from a sputum sample, and the sample is diluted in a buffer solution, centrifuged at a low speed to reduce the level of particulate matter in the sample, and the sample is prepared following Protocol 8-2.
The MRSA strain is spiked into a solution containing a whole blood, plasma, serum sample, and the sample is diluted in a buffer solution and centrifuged at a low speed (in the case of whole blood) to reduce the level of particulate matter in the sample. A sample is then prepared following Protocol 8-2, except that in place of spiking 10 μl of the diluted bacterial colony onto a Clinical Swab, 10 μl of the diluted bacterial colony is spiked directly into the tube containing the blood sample.
The MRSA strain is spiked into a solution containing a homogenized groin skin sample or other tissue sample. The DNA is then extracted and the sample prepared using a commercial kit for extraction of DNA from tissue samples, such as the D-Tail kit (Syntezza Bioscience Ltd., Jerusalem, Israel).

### Protocol 8-1: Preparation of the Direct Bacterial DNA Samples

A sample of the diluted bacterial colony is removed and pipetted into a test tube containing lysis buffer. The tube is then incubated in a heating block. The RNaseH2 Mixture is added and then an aliquot of the resulting solution is then transferred into a PCR Reaction Tube containing a qPCR reaction mixture.

Protocol 8-2: Preparation of Bacterial DNA Samples Spiked onto Clinical Swabs.

Bacterial DNA samples spiked onto clinical swabs are prepared as described in the previous Example.

### Results

As seen in Example 3 for Spiked Nasal samples from MRSA Strain #3, assays targeting the orfX region and internal control exhibit PCR amplification. However, the assays targeting the nuc and mecA genes exhibit PCR inhibition. These results demonstrate that PCR-inhibiting factors are present in a number of clinical specimens.

Moreover, these PCR-inhibiting factors are shown to adversely affect the performance of hot-start primer-only assays that exhibit weak primer-target hybridization, such as the nuc and mecA assays exemplified herein, but not hot-start primer-only assays that exhibit strong primer-target hybridization, such as the orfX and internal control targeting assays exemplified herein.

### Example 9

### A Combination of Hot-Start Primers and Regular Primers Overcomes Inhibition of qPCR by Clinical Specimens

The experiment of the previous Example is repeated, in this case using hot-start primers exclusively for the OrfX region and the IC, and a mixture of hot-start and non-hot-start primers for the nuc and mecA genes. Use of this primer mixture enables efficient amplification of all 4 targets, even in clinical specimens shown in the previous Example to inhibit certain hot-start PCR reactions.

1. A PCR reaction mixture comprising:
a) a first set of primers for amplifying a first target polynucleotide sequence;
b) a second set of primers for amplifying a second target polynucleotide sequence; and
c) an activating enzyme,
wherein the primers that hybridize to at least one end of said first target polynucleotide sequence are a mixture of hot-start primers and non-hot-start primers, wherein the reaction product of amplifying said first target polynucleotide sequence using said non-hot-start primers is a substrate for amplification using said hot-start primers; and
wherein said hot-start primers contain an inactivating chemical modification that is reversed by the action of said activating enzyme, wherein said hot-start primers become a substrate for said activating enzyme when said hot-start primers are hybridized to a complementary sequence at elevated temperatures.

2. The PCR reaction mixture of claim 1, wherein the sequences recognized by said non-hot-start primers flank the sequences recognized by said hot-start primers.

3. The PCR reaction mixture of claim 1, wherein the sequences recognized by said non-hot-start primers overlap or are identical to the sequences recognized by said hot-start primers.

4. The PCR reaction mixture of claim 1, wherein the primers that hybridize to only one end of said first target polynucleotide sequence are said mixture of hot-start primers and non-hot-start primers.

5. The PCR reaction mixture of claim 1, wherein the primers that hybridize to both ends of said first target polynucleotide sequence are said mixture of hot-start primers and non-hot-start primers.

6. The PCR reaction mixture of claim 1, wherein the ratio of said hot-start primers to said non-hot-start primers is between 1:9 and 19:1, inclusive.

7. The PCR reaction mixture of claim 1, wherein said inactivating chemical modification is a 3′ blocking group.

8. The PCR reaction mixture of claim 1, wherein said activating enzyme is a thermostable RNase H enzyme.

9. The PCR reaction mixture of claim 1, wherein the primers that hybridize to at least one end of said second target polynucleotide sequence are a mixture of hot-start primers, as defined in claim 1, and non-hot-start primers, wherein the reaction product of amplifying said second target polynucleotide sequence using said non-hot-start primers is a substrate for amplification using said hot-start primers.

10. The PCR reaction mixture of claim 1, further comprising a third set of primers for amplifying a third target polynucleotide sequence.

11. The PCR reaction mixture of claim 10, wherein the primers that hybridize to at least one end of said third target polynucleotide sequence are a mixture of hot-start primers, as defined in claim 1, and non-hot-start primers, wherein the reaction product of amplifying said third target polynucleotide sequence using said non-hot-start primers is a substrate for amplification using said hot-start primers.

12. The PCR reaction mixture of claim 1, further comprising a probe suitable for real-time PCR.

13. A kit comprising the PCR reaction mixture of claim 1 and instructions for use thereof.

14. The kit of claim 13, wherein said kit is indicated for detecting a target polynucleotide sequence or DNA marker in a test sample.

15. A method of detecting a target polynucleotide sequence in a test sample, said method comprising the steps of performing real-time PCR on nucleic acids from said test sample using the PCR reaction mixture of claim 12; measuring the signal generated by amplification of said target polynucleotide sequence; and comparing said signal to a reference standard,
whereby, if said signal is equal to or below said reference standard, then said target polynucleotide sequence is present in said test sample.

16. A method of detecting a first target polynucleotide sequence and a second target polynucleotide sequence in a test sample, wherein the nucleic acids of said test sample have been derived from a clinical specimen and have not been extensively purified, the method comprising the steps of:
(a) amplifying nucleic acids from said test sample using a PCR reaction mixture; and
(b) detecting the amplified PCR product, wherein the PCR reaction mixture comprises: i) a first set of primers, comprising at least one forward primer and at least one reverse primer, that is capable of amplifying said first target polynucleotide sequence; and ii) a second set of primers, comprising at least one forward primer and at least one reverse primer, that is capable of amplifying said second target polynucleotide sequence, wherein at least one of said forward primer and said reverse primer of said first set of primers is provided as a mixture of hot-start primers and non-hot-start primers.

17. The method of claim 16, wherein said clinical specimen is a body fluid.

18. The method of claim 17, wherein said body fluid is selected from nasal fluid, vaginal secretions, whole blood, serum, plasma, cerebrospinal fluid, urine, lymph fluids, tears, saliva, milk, amniotic fluid, and an external secretion of the respiratory, intestinal or genitourinary tract.

19. The method of claim 16, wherein said clinical specimen is a tissue.

20. The method of claim 16, wherein the sequences recognized by said non-hot-start primers flank the sequences recognized by said hot-start primers.

21. The method of claim 16, wherein the sequences recognized by said non-hot-start primers overlap or are identical to the sequences recognized by said hot-start primers.

22. The method of claim 16, wherein the primers that hybridize to only one end of said first target polynucleotide sequence are said mixture of hot-start primers and non-hot-start primers.

23. The method of claim 16, wherein the primers that hybridize to both ends of said first target polynucleotide sequence are said mixture of hot-start primers and non-hot-start primers.

24. The method of claim 16, wherein the ratio of said hot-start primers to said non-hot-start primers is between 1:9 and 19:1, inclusive.

25. The method of claim 16, wherein said PCR reaction mixture further comprises a probe suitable for real-time PCR.
